SEQUENCE LISTING (71 sequences)

SEQ ID NO: 1 - DNA, 22 bp, Artificial Sequence
  Oligonucleotide
tcgtcgttttgcgcggccgccg

SEQ ID NO: 2 - DNA, 21 bp, Artificial Sequence
  Oligonucleotide
ggggacgacgtcgtggggggg

SEQ ID NO: 3 - DNA, 15 bp, Artificial Sequence
  Oligonucleotide
tcgacgtcgtggggg

SEQ ID NO: 4 - DNA, 24 bp, Artificial Sequence
  Oligonucleotide
tcgtcgttttgtcgttttgtcgtt

SEQ ID NO: 5 - DNA, 20 bp, Artificial Sequence
  Oligonucleotide
tccaggacttctctcaggtt

SEQ ID NO: 6 - DNA, 22 bp, Artificial Sequence
  Oligonucleotide
tcgtcgttttcggcgcgcgccg

SEQ ID NO: 7 - DNA, 14 bp, Artificial Sequence
  Oligonucleotide
tcgtcgacgtgggg

SEQ ID NO: 8 - DNA, 15 bp, Artificial Sequence
  Oligonucleotide
tcgccggcgtggggg

SEQ ID NO: 9 - DNA, 15 bp, Artificial Sequence
  Oligonucleotide
tcggcgccgtggggg

SEQ ID NO: 10 - DNA, 21 bp, Artificial Sequence
  Oligonucleotide
tcgacgtcgacgtcgtggggg

SEQ ID NO: 11 - DNA, 16 bp, Artificial Sequence
  Oligonucleotide
tcgacgtcgttggggg

SEQ ID NO: 12 - DNA, 16 bp, Artificial Sequence
  Oligonucleotide
gtcgacgtcgtggggg

SEQ ID NO: 13 - DNA, 17 bp, Artificial Sequence
  Oligonucleotide
gtcgacgtcgttggggg

SEQ ID NO: 14 - DNA, 16 bp, Artificial Sequence
  Oligonucleotide
tcgtcgacgttggggg

SEQ ID NO: 15 - DNA, 15 bp, Artificial Sequence
  Oligonucleotide
tcgacgtcgtgggng

SEQ ID NO: 16 - DNA, 15 bp, Artificial Sequence
  Oligonucleotide
tcgacgtcgtgnnnn

SEQ ID NO: 17 - DNA, 15 bp, Artificial Sequence
  Oligonucleotide
tcgacgtcgtggggg

SEQ ID NO: 18 - DNA, 10 bp, Artificial Sequence
  Oligonucleotide
tcgacgtcgt

SEQ ID NO: 19 - DNA, 24 bp, Artificial Sequence
  Oligonucleotide
acgacgttttgtcgttttgtcgtt

SEQ ID NO: 20 - DNA, 10 bp, Artificial Sequence
  Oligonucleotide
acgacgtcgt

SEQ ID NO: 21 - DNA, 20 bp, Artificial Sequence
  Oligonucleotide
acgacgtcgttttttttttt

SEQ ID NO: 22 - DNA, 20 bp, Artificial Sequence
  Oligonucleotide
acgacgtcgttttttttttt

SEQ ID NO: 23 - DNA, 21 bp, Artificial Sequence
  Oligonucleotide
ggggtcgacgtcgtggggggg

SEQ ID NO: 24 - DNA, 19 bp, Artificial Sequence
  Oligonucleotide
ggggtcgacgtcgtggggg

SEQ ID NO: 25 - DNA, 21 bp, Artificial Sequence
  Oligonucleotide
ggggtcgtcgtcgtggggggg

SEQ ID NO: 26 - DNA, 16 bp, Artificial Sequence
  Oligonucleotide
gggtgggtgggtgggt

SEQ ID NO: 27 - DNA, 20 bp, Artificial Sequence
  Oligonucleotide
ggcgtggcgtggcgtggcgt

SEQ ID NO: 28 - DNA, 23 bp, Artificial Sequence
  Oligonucleotide
ggcgtcggcgtcggcgtcggcgt

SEQ ID NO: 29 - DNA, 15 bp, Artificial Sequence
  Oligonucleotide
ncgacgtcgtggggg

SEQ ID NO: 30 - DNA, 16 bp, Artificial Sequence
  Oligonucleotide
tcgacgtcgtgggggt

SEQ ID NO: 31 - DNA, 27 bp, Artificial Sequence
  Oligonucleotide
tcgacgtcgtddddtcgacgtcgtddd

SEQ ID NO: 32 - DNA, 15 bp, Artificial Sequence
  Oligonucleotide
tcgacgtcgtggggg

SEQ ID NO: 33 - DNA, 15 bp, Artificial Sequence
  Oligonucleotide
tcgacgtcgtggggg

SEQ ID NO: 34 - DNA, 15 bp, Artificial Sequence
  Oligonucleotide
acgacgtcgtggggg

SEQ ID NO: 35 - DNA, 15 bp, Artificial Sequence
  Oligonucleotide
tcgacgacgtggggg

SEQ ID NO: 36 - DNA, 15 bp, Artificial Sequence
  Oligonucleotide
acgtcgtcgtggggg

SEQ ID NO: 37 - DNA, 15 bp, Artificial Sequence
  Oligonucleotide
tcgacgtcgtcgggg

SEQ ID NO: 38 - DNA, 15 bp, Artificial Sequence
  Oligonucleotide
tcgacgtcgtggtgg

SEQ ID NO: 39 - DNA, 14 bp, Artificial Sequence
  Oligonucleotide
tcgacgtcgtgggg

SEQ ID NO: 40 - DNA, 10 bp, Artificial Sequence
  Oligonucleotide
tcgacgtcgt

SEQ ID NO: 41 - DNA, 10 bp, Artificial Sequence
  Oligonucleotide
tcgacgtcgt

SEQ ID NO: 42 - DNA, 10 bp, Artificial Sequence
  Oligonucleotide
tcgacgtcgt

SEQ ID NO: 43 - DNA, 10 bp, Artificial Sequence
  Oligonucleotide
tcgacgtcgt

SEQ ID NO: 44 - DNA, 10 bp, Artificial Sequence
  Oligonucleotide
tcgacgtcgt

SEQ ID NO: 45 - DNA, 15 bp, Artificial Sequence
  Oligonucleotide
tcgacgtcgtggggg

SEQ ID NO: 46 - DNA, 15 bp, Artificial Sequence
  Oligonucleotide
tcgacgtcgtggggt

SEQ ID NO: 47 - DNA, 15 bp, Artificial Sequence
  Oligonucleotide
tcgacgtcgaggggg

SEQ ID NO: 48 - DNA, 15 bp, Artificial Sequence
  Oligonucleotide
tcgacgtcgaggggt

SEQ ID NO: 49 - DNA, 14 bp, Artificial Sequence
  Oligonucleotide
tcgacgtcgagggg

SEQ ID NO: 50 - DNA, 10 bp, Artificial Sequence
  Oligonucleotide
tcgacgtcga

SEQ ID NO: 51 - DNA, 24 bp, Artificial Sequence
  Oligonucleotide
tgctgcttttgtgcttttgtgctt

SEQ ID NO: 52 - DNA, 10 bp, Artificial Sequence
  Oligonucleotide
tcgacgtcgt

SEQ ID NO: 53 - DNA, 16 bp, Artificial Sequence
  Oligonucleotide
tcgtcgtttcgtcgtt

SEQ ID NO: 54 - DNA, 24 bp, Artificial Sequence
  Oligonucleotide
acgacgtcgtttttacgacgtcgt

SEQ ID NO: 55 - DNA, 16 bp, Artificial Sequence
  Oligonucleotide
tcgtcgtttcgtcgtt

SEQ ID NO: 56 - DNA, 24 bp, Artificial Sequence
  Oligonucleotide
acgacgtcgtttttacgacgtcgt

SEQ ID NO: 57 - DNA, 16 bp, Artificial Sequence
  Oligonucleotide
tcgtcgtttcgtcgtt

SEQ ID NO: 58 - DNA, 24 bp, Artificial Sequence
  Oligonucleotide
tcgtcgttttgtcgttttgtcgtt

SEQ ID NO: 59 - DNA, 24 bp, Artificial Sequence
  Oligonucleotide
tcgtcgttttgtcgttttgtcgtt

SEQ ID NO: 60 - DNA, 24 bp, Artificial Sequence
  Oligonucleotide
tcgtcgttttgtcgttttgtcgtt

SEQ ID NO: 61 - DNA, 24 bp, Artificial Sequence
  Oligonucleotide
tcgtcgttttgtcgttttgtcgtt

SEQ ID NO: 62 - DNA, 24 bp, Artificial Sequence
  Oligonucleotide
tcgtcgttttgtcgttttgtcgtt

SEQ ID NO: 63 - DNA, 22 bp, Artificial Sequence
  Oligonucleotide
tcgtcgttttcggcggccgccg

SEQ ID NO: 64 - DNA, 10 bp, Artificial Sequence
  Oligonucleotide
acgacgtcgt

SEQ ID NO: 65 - DNA, 10 bp, Artificial Sequence
  Oligonucleotide
acgacgtcgt

SEQ ID NO: 66 - DNA, 27 bp, Artificial Sequence
  Oligonucleotide
acgacgtcgtddddacgacgtcgtddd

SEQ ID NO: 67 - DNA, 30 bp, Artificial Sequence
  Oligonucleotide
dddacgacgtcgtddddacgacgtcgtddd

SEQ ID NO: 68 - DNA, 23 bp, Artificial Sequence
  Oligonucleotide
tcgtcgacgttcggcgcgcgccg

SEQ ID NO: 69 - DNA, 23 bp, Artificial Sequence
  Oligonucleotide
tcgtcgacgatcggcgcgcgccg

SEQ ID NO: 70 - DNA, 49 bp, Artificial Sequence
  Oligonucleotide
nnnnnnnnnnhnnnnnnnnnnnnnnnnnnggggggggggnnnnnnnnnn

SEQ ID NO: 71 - DNA, 29 bp, Artificial Sequence
  Oligonucleotide
nnnnnnnnnnhnnnnnnnnnnnnnnnnnn

(19) United States
(12) Patent Application Publication
(10) Pub. No.: US 2010/0285041 A1
(43) Pub. Date: Nov. 11, 2010

(54) Class A Oligonucleotides with Immunostimulatory Potency

(76) Inventors: Eugen Uhlmann, Glashuetten (DE); Jörg Vollmer, Duesseldorf (DE); Arthur Mertz Kreig, Wellesley, MA (US)

Correspondence Address: WOLF GREENFIELD & SACKS, P.C., 600 ATLANTIC AVENUE, BOSTON, MA, 02210-2206, US

(21) Appl. No.: 12/600,364

(22) PCT Filed: May 15, 2008
(86) PCT No.: PCT/IB08/01199

§ 371 Date: Nov. 16, 2009

Related U.S. Application Data
(60) Provisional application No. 60/930,580, filed on May 17, 2007.

(51) Int. Cl. A61K 39/00; C07H 21/04; A61P 35/00; A61P 31/00; A61P 37/08; A61P 37/04; A61P 11/06

(52) U.S. Cl. 424/184.1; 536/23.1

(57) ABSTRACT

The invention provides an immunostimulatory nucleic acid comprising CpG motifs, and methods of use thereof in stimulating immunity.

### FIELD OF THE INVENTION

The present invention relates to the induction of an immune response, specifically to immunostimulatory oligonucleotides and their use in inducing an immune response.

### INTRODUCTION

Bacterial DNA has immune stimulatory effects to activate B cells and natural killer cells, but vertebrate DNA does not (Tokunaga, T., et al., 1988. Jpn. J. Cancer Res. 79:682-686; Tokunaga, T., et al., 1984, JNCI 72:955-962; Messina, J. P., et al., 1991, J. Immunol. 147:1759-1764; and reviewed in Krieg, 1998, In: Applied Oligonucleotide Technology, C. A. Stein and A. M. Krieg, (Eds.), John Wiley and Sons, Inc., New York, N.Y., pp. 431-448). It is now understood that these immune stimulatory effects of bacterial DNA are a result of the presence of unmethylated CpG dinucleotides in particular base contexts (CpG motifs), which are common in bacterial DNA, but methylated and underrepresented in vertebrate DNA (Krieg et al, 1995 Nature 374:546-549; Krieg, 1999 Biochim. Biophys. Acta 93321:1-10). The immune stimulatory effects of bacterial DNA can be mimicked with synthetic oligodeoxynucleotides (ODN) containing these CpG motifs. Such CpG ODN have highly stimulatory effects on human and murine leukocytes, inducing B cell proliferation; cytokine and immunoglobulin secretion; natural killer (NK) cell lytic activity and IFN-γ secretion; and activation of dendritic cells (DCs) and other antigen presenting cells to express costimulatory molecules and secrete cytokines, especially the Th1-like cytokines that are important in promoting the development of Th1-like T cell responses. These immune stimulatory effects of native phosphodiester backbone CpG ODN are highly CpG specific in that the effects are dramatically reduced if the CpG motif is methylated, changed to a GpC, or otherwise eliminated or altered (Krieg et al, 1995 Nature 374:546-549; Hartmann et al, 1999 Proc. Natl. Acad. Sci. USA 96:9305-10). The strong, yet balanced, cellular and humoral immune responses that result from CpG stimulation reflect the body's own natural defense system against invading pathogens and cancerous cells. Thus, CpG containing oligonucleotides, relying on this innate immune defense mechanism, can utilize a unique and natural pathway for immune therapy. They can thereby be used to treat cancer, infectious diseases, allergy, asthma and other disorders, and to help protect against opportunistic infections following cancer chemotherapies.

Several different classes of CpG oligonucleotides have recently been described. One class is potent for activating B cells but is relatively weak in inducing IFN-α and NK cell activation; this class has been termed the B-class. The B-class CpG oligonucleotides typically are fully stabilized and include an unmethylated CpG dinucleotide within certain preferred base contexts. See, e.g., U.S. Pat. Nos. 6,194,388; 6,207,646; 6,214,806; 6,218,371; 6,239,116; and 6,339,068. Another class of CpG oligonucleotides activates B cells and NK cells and induces IFN-α; this class has been termed the C-class. The C-class CpG oligonucleotides, as first characterized, typically are fully stabilized, include a B-class-type sequence and a GC-rich palindrome or near-palindrome. This class has been described in U.S. patent application Ser. No. 10/224,523 filed on Aug. 19, 2002 and related PCT Patent Application PCT/US02/26468 published under International Publication Number WO 03/015711. A third class is the A-class. A-class CpG immunostimulatory oligonucleotides have been described in U.S. Pat. No. 6,949,520 and PCT application PCT/US00/26527 published under International Publication Number WO 01/22990, both filed on Sep. 27, 2000, the contents of which are hereby incorporated by reference. These oligonucleotides are characterized by the ability to induce high levels of interferon-α while having minimal effects on B cell activation.

### SUMMARY

In one aspect the invention provides a use of a modified A-class oligonucleotide of the invention for the preparation of a medicament for treating cancer, infectious disease, asthma, allergy, allergic rhinitis, or autoimmune disease in a subject.

In one aspect the invention provides a composition useful for the treatment of cancer, infectious disease, asthma, allergy, allergic rhinitis, or autoimmune disease. The composition according to this aspect includes a modified A-class oligonucleotide of the invention and a cancer, infectious disease, asthma, allergy, allergic rhinitis, or autoimmune disease medicament or agent.

Use of an oligonucleotide of the invention for stimulating an immune response is also provided as an aspect of the invention.

One aspect of the invention is an immunostimulatory oligonucleotide of the formula

(SEQ ID NO: 70)
5′-(Z₁)_{K}X₁Y₁R₁X₂Y₂R₂X₃Y₃R₃(Z₂)_{L}(G)_{N}(Z₃)_{M}-3′

where X₁ is any nucleotide except deoxyguanosine (dG), X₂ and X₃ are any nucleotide, Y₁, Y₂, and Y₃ are deoxycyticine (dC), 5-methyl-dC, 5-hydroxy-dC or 5-fluoro-dC, R₁, R₂ and R₃ are dG, deoxyinosine (dI), 6-Thio-dG, or 7-deaza-dG, and Z₁, Z₂ and Z₃ are any nucleotide, and wherein K, L, and M each independently represent 0-10, N is 4-10 and where the immunostimulatory oligonucleotide is less than 16 nucleotides in length. In one embodiment X₁ is T, dU, dI, or dA. In another embodiment, X₂ is T, dU, dA, or 7-deaza-dA. In yet another embodiment, X₃ is T, dU, dA, or 7-deaza-dA. In still another embodiment, Z₁ is dG, dT, dU, dI, or 7-deaza-dG. In one embodiment Z₂ is T. In another embodiment Z₃ is T. In one embodiment the immunostimulatory oligonucleotide comprises fewer than six phosphorothioate linkages. In another embodiment the immunostimulatory oligonucleotide comprises four phosphorothioate linkages. In one embodiment X₂ and X₃ are complementary nucleotides. In another embodiment the sequence Y₁R₁X₂Y₂R₂X₃Y₃R₃ forms a palindrome or near-palindrome. In one embodiment K represents 0-10 nucleotides. In another embodiment K represents 0-2 nucleotides. In yet another embodiment L represents 0-10 nucleotides. In still another embodiment L represents 0-2 nucleotides. In one embodiment M represents 0-10 nucleotides. In another embodiment M represents 0-2 nucleotides. In one embodiment N represents 2-40 nucleotides. In another embodiment N represents 5 nucleotides. In yet another embodiment N represents 4 nucleotides.

In one embodiment the immunostimulatory oligonucleotide comprises a palindromic domain of at least 6 and less than 11 nucleotides in length and including at least 3 YR dinucleotides having phosphodiester or phosphodiester-like internucleotide linkages, wherein Y is dC, 5-methyl-dC, 5-hydroxy-dC or 5-fluoro-dC, and R is dG, dI, 6-Thio-dG, or 7-deaza-dG, linked to a Poly G domain, either directly or indirectly, wherein the Poly G domain includes at least 3 and less than 8 consecutive Gs, wherein when the palindromic domain is indirectly linked to the Poly-G domain, the indirect linkage is comprised of a nucleotide sequence of 1-10 nucleotides or a non-nucleotide linker, wherein the oligonucleotide has a length of less than 18 nucleotides. In another embodiment, the oligonucleotide includes at least 2 and less than 6 stabilized internucleotide linkages. In yet another embodiment, the oligonucleotide has 4 stabilized internucleotide linkages. In one embodiment the stabilized internucleotide linkages are phosphorothioate linkages. In another embodiment the oligonucleotide does not include a 5′ GG. In one embodiment the nucleotide of the palindromic domain has a phosphodiester internucleotide linkage. In another embodiment the palindromic domain has less than 9 nucleotides. In yet another embodiment the oligonucleotide includes one or more nucleotide 5′ to the palindromic domain.

In one embodiment the immunostimulatory oligonucleotide comprises a palindromic domain of at least 6 and less than 11 nucleotides in length and including at least 3 Y′R′ dinucleotides having phosphodiester or phosphodiester-like internucleotide linkages, wherein Y′ is 5-methyl-dC, 5-hydroxy-dC or 5-fluoro-dC, and R is dI, dG, 6-Thio-dG, or 7-deaza-dG, linked to a Poly G domain, either directly or indirectly, wherein the Poly G domain includes at least 3 and less than 8 consecutive Gs, wherein when the palindromic domain is indirectly linked to the Poly-G domain, the indirect linkage is comprised of a nucleotide sequence of 1-10 nucleotides or a non-nucleotide linker.

Another aspect of the invention is an immunostimulatory oligonucleotide of the formula

(SEQ ID NO: 71)
5′-(Z₁)_{K}X₁Y₁R₁X₂Y₂R₂X₃Y₃R₃(Z₂)_{L}Q-3′

wherein X₁ is any nucleotide except dG, X₂ and X₃ are any nucleotide, Y₁, Y₂, and Y₃ are dC, 5-methyl-dC, 5-hydroxy-dC or 5-fluoro-dC, R₁, R₂ and R₃ are dG, dI, 6-Thio-dG, or 7-deaza-dG, and Z₁ and Z₂ are any nucleotide, and Q is a lipophilic moiety, and wherein K and L each independently represent 0-10, and wherein the immunostimulatory oligonucleotide is less than 16 nucleotides in length.

In another aspect of the invention the immunostimulatory oligonucleotides are useful as compositions comprising any of the immunostimulatory oligonucleotides of the instant invention together with a pharmaceutical carrier. In one embodiment the immunostimulatory oligonucleotide is SEQ ID NO:3, SEQ ID NO:4, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:29, SEQ ID NO:30, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, OR SEQ ID NO:43.

Another aspect of the invention is a method of stimulating an immune response in a subject by administering to a subject in need of such treatment any of the compositions of the instant invention. In one embodiment the subject in need has or is at risk of having cancer, infectious disease, asthma, allergy, allergic rhinitis, or autoimmune disease. In another embodiment the subject has previously been unresponsive to conventional therapeutic treatments. In yet another embodiment the composition is administered intravenously. In still another embodiment the composition is administered subcutaneously. In one embodiment the subject is a subject having or at risk of having an infectious disease. In another embodiment the infectious disease is a viral disease. In yet another embodiment the viral disease is Hepatitis B, Hepatitis C, Cytomegalovirus, (CMV), Papilloma Virus, HIV or Herpes simplex viruses (HSV). In still another embodiment the infectious disease is Leishmania, Listeria, or Anthrax. In another embodiment the subject is a subject undergoing anti-cancer treatment. In another embodiment the anti-cancer treatment is radiation, chemotherapy, a vaccine chemotherapy, a vaccine (e.g., an in vitro primed dendritic cell vaccine or a cancer antigen vaccine), or an antibody based therapy. In another embodiment the subject is a subject being treated with an anti-viral medicament.

In one aspect the invention provides a method of treating a subject having a cancer, infectious disease, asthma, allergy, allergic rhinitis, or autoimmune disease. The method according to this aspect of the invention includes the step of administering to a subject having a cancer, infectious disease, asthma, allergy, allergic rhinitis, or autoimmune disease an effective amount of the composition of the invention and an anti-cancer, infectious disease, asthma, allergy, allergic rhinitis, or autoimmune disease therapy to treat the subject.

A method for manufacturing a medicament of an oligonucleotide of the invention for stimulating an immune response is also provided.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF THE DRAWINGS

The figures are illustrative only and are not required for enablement of the invention disclosed herein.

FIG. 1 is five graphs demonstrating induction of IFN-α by a shortened A-class oligonucleotide, SEQ ID NO:3. The activity is compared to that of the longer A-class oligonucleotide from which it is derived (SEQ ID NO:2), as well as B-class ODN (SEQ ID NO:4), C-class ODN (SEQ ID NO:1 and 68), P-class ODN (SEQ ID NO:69) and negative control ODN (SEQ ID NO:5). In FIGS. 1a-1d the y-axes represent IFN-α in pg/ml and the x-axes represent ODN concentration in μM. FIG. 1e shows a comparison of the ability of the oligos to stimulate TLR9 activity. The y-axis represents stimulation index and the x-axis represents ODN concentration in 10^{x} μM.

FIG. 2 is two graphs demonstrating the induction of IFN-α (FIG. 2a) and IP-10 (FIG. 2b) by a number of SEQ ID NO:3 derivatives (SEQ ID NO:32-39) as measured by ELISA assay. The y-axes are cytokine concentration and the x-axes are ODN concentration in μM.

FIG. 3 is six graphs demonstrating the induction of IFN-α (FIGS. 3a-3c) and IP-10 (FIGS. 3d-3f) by a number of SEQ ID NO:3 derivatives (SEQ ID NO:7-31) as measured by ELISA assay. The y-axes are cytokine concentration and the x-axes are ODN concentration in μM.

FIG. 4 is a drawing describing the process for making lipophilic ODN derivatives with either hexadecyl glyceryl ether or triethylene glycol in place of the 3′ poly G motif.

FIG. 5 is a graph showing the activity of two derivatives of SEQ ID NO:3, SEQ ID NO:40 with a hexadecyl glyceryl ether moiety and SEQ ID NO:41 with a triethylene glycol moiety. SEQ ID NO:52 is a control ODN of the same sequence but no lipophilic moiety. The activity is also compared to a conventional A-class ODN (SEQ ID NO:2) and a negative control ODN (SEQ ID NO:5). The y-axis is IFN-α concentration in pg/ml and the x-axis is ODN concentration in μM.

FIG. 6 is a drawing illustrating the structure of lipophilic ODN derivatives with cholesterol.

FIG. 7 is three graphs showing the activity of the two derivatives of SEQ ID NO:3 shown in FIG. 5 data but with cholesterol moieties in place of the 3′ poly G motif. SEQ ID NO:43 has a phosphodiester backbone and a 3′ cholesterol tag, whereas SEQ ID NO:42 is stabilized by phosphorothioate bonds at the terminal linkages and a 3′ cholesterol. SEQ ID NO:44 has a phosphodiester backbone and a cholesterol tag on both the 3′ and 5′ ends. FIGS. 7a and 7b show IFN-α induction. The activity is also compared to a conventional A-class ODN (SEQ ID NO:2), a B-class ODN (SEQ ID NO:4), another shortened A-class ODN (SEQ ID NO:3), and a negative control ODN (SEQ ID NO:5). FIG. 7c shows IL-10 induction. The y-axes are cytokine, concentration and the x-axes are ODN concentration in μM.

FIG. 8 is four graphs showing the ability of SEQ ID NO:3 to induce of IP-10 in vivo by various routes of administration. Balb/c mice were injected subcutaneous (SC), intravenous (IV), or intra-peritoneal (IP) with 500 μg of the indicated ODN and bled at 3 hours (solid bars), or intra-pulmonary with 250 μg of the indicated ODN and bled at 8 hours (hatched bars). The y-axes are IP-10 concentration in ng/ml and the x-axes represent ODN used.

FIG. 9 is four graphs showing the ability of SEQ ID NO:3 to induce of IL-12 in vivo by various routes of administration. Balb/c mice were injected SC, IV, or IP with 500 μg of the indicated ODN and bled at 3 hours (solid bars), or intra-pulmonary with 250 μg of the indicated ODN and bled at 8 hours (hatched bars). The y-axes are IL-12 concentration in ng/ml and the x-axes represent ODN used.

FIG. 10 is four graphs showing the ability of SEQ ID NO:3 to induce of IL-6 in vivo by various routes of administration. This activity was compared to that of a B-class ODN (SEQ ID NO:4), a conventional A-class ODN (SEQ ID NO:2), a short cholesterol-modified ODN (SEQ ID NO:50) and a control ODN (SEQ ID NO:51). Balb/c mice were injected SC, IV, or IP with 500 μg of the indicated ODN and bled at 3 hours (solid bars), or intra-pulmonary with 250 μg of the indicated ODN and bled at 8 hours (hatched bars). The y-axes are IL-6 concentration in ng/ml and the x-axes represent ODN used.

### DETAILED DESCRIPTION

The invention in one aspect involves the finding that a specific sub-class of immunostimulatory oligonucleotide is highly effective in mediating immune stimulatory effects. These oligonucleotides are useful therapeutically and prophylactically for stimulating the immune system to treat cancer, infectious diseases, allergy, asthma and other disorders.

A-Class immunostimulatory CpG oligonucleotides, such as oligonucleotide SEQ ID NO:2, are characterized by their very efficient induction of IFN-α secretion, but low B cell stimulation. SEQ ID NO:2 is composed of a palindromic phosphodiester CpG sequence clamped by phosphorothioate (G)n stretches: G*G*G-G-A-C-G-A-C-G-T-C-G-T-G-G*G*G*G*G*G (SEQ ID NO:2). (* is phosphorothioate, - is phosphodiester) A-Class oligonucleotides, in which the 3′- and 5′-ends are phosphorothioate-modified and the center portion is phosphodiester, have runs of at least four G residues at both ends of the oligonucleotide. As a result of intermolecular tetrad formation which results in high molecular weight aggregates, the development of G-rich oligonucleotides has been difficult. Issues related to the biophysical properties of this class of compounds include tendency to aggregation, poor solubility, difficulty in quality control and solid phase extraction (SPE) used in PK studies.

It is known that (G)n stretches in oligonucleotides, where n≧4, lead to intermolecular tetrad formation resulting in non homogeneous high molecular weight aggregates. The uptake of oligonucleotides with (G)n stretches is about 20 to 40-times higher than of non-aggregated oligonucleotides and the intracellular localization appears also to be different. It is not understood how these observations correlate with biological activity.

In an attempt to discover new immunostimulatory oligonucleotides having similar potency to A-class oligonucleotides, such as SEQ ID NO:2, but more favorable biophysical properties, a series of oligonucleotides with only 3′ (G)n stretches was developed according to the invention. These modified A-class oligonucleotides can form the intramolecular tetrads responsible for enhanced uptake by cells, but not higher molecular weight aggregates. Thus, they show improved solubility under biologically relevant conditions. Oligonucleotides with a 5′TCG motif are usually recognized by TLR9; therefore new palindromes were designed to include a 5′TCG TLR9 recognition sequence. This in turn allows for multiple TLR9 recognition sequences per intermolecular tetrad. These oligonucleotides also may have fewer stabilized internucleotide linkages, which may increase their ability to stimulate TLR9 activity.

Thus, the invention involves, in one aspect, the discovery that a sub-class of A-class oligonucleotides referred to herein as “modified A-class” oligonucleotides, with a shortened palindrome sequence, fewer phosphorothioate residues, and no 5′ G-rich domain. Exemplary modified A-class oligonucleotides are presented in table I (below). Surprisingly, these modified A-class oligonucleotides, e.g. SEQ ID NO:3, showed as high or higher levels of IFN-α induction than the classical A-class oligonucleotide SEQ ID NO:2, from which its sequence is derived. The immunostimulatory modified A-class oligonucleotides of the instant invention are described by formula I:

(SEQ ID NO: 70)
5′-(Z₁)_{K}X₁Y₁R₁X₂Y₂R₂X₃Y₃R₃(Z₂)_{L}(G)_{N}(Z₃)_{M}-3′

where X₁ is any nucleotide except deoxyguanosine (dG), X₂ and X₃ are any nucleotide, Y₁, Y₂, and Y₃ are deoxycyticine or a modified deoxycyticine (dC) and R₁, R₂ and R₃ are deoxyguanosine or a modified deoxyguanosine. Thus, a YR dinucleotide can be a CG (CpG) dinucleotide. Z₁, Z₂ and Z₃ are any nucleotide; K, L, and M each independently represent 0-10 nucleotides and can be any nucleotide, and N is 4-10 nucleotides.

In one embodiment X₁ is T, deoxyuracil (dU), deoxyinosine (I), or deoxyadenine (dA). In another embodiment, X₂ is T, dU, dA, or 7-deaza-dA. In yet another embodiment, X₃ is T, dU, dA, or 7-deaza-dA. In another embodiment, Z₁ is dG, dT, dU, dI, or 7-deaza-dG. In one embodiment Z₂ is T. In another embodiment Z₃ is T. The immunostimulatory oligonucleotides typically contain 6 or fewer phosphorothioate linkages, but are not so limited. In one embodiment X₂ and X₃ are complementary nucleotides.

In one embodiment the immunostimulatory oligonucleotide comprises a palindromic domain of at least 6 and less than 11 nucleotides in length. A “palindromic domain” shall mean a domain containing an inverted repeat, i.e., a sequence such as ABCDEE′D′C′B′A′ in which A and A′, B and B′, C and C′, D and D′, and E and E′ are bases capable of forming the usual Watson-Crick base pairs. Such a sequence is referred to herein as a “palindrome”. In some embodiments the palindromic domain contains a near-palindrome rather than a palindrome. A “near-palindrome” as used herein refers to a sequence that is not a perfect palindromic sequence. In vivo, palindromic and near palindromic sequences may form double-stranded structures. In one embodiment the sequence Y₁R₁X₂Y₂R₂X₃Y₃R₃ forms a palindrome or near-palindrome. The sequence of the palindrome or near-palindrome in some embodiments may include at least 3 YR dinucleotides having phosphodiester or phosphodiester-like internucleotide linkages. In some embodiments the internucleotide linkages of the palindromic or near-palindromic domain are phosphodiester linkages. The palindrome or near-palindrome sequence may occur at the extreme 5′ end of the oligonucleotide. Alternatively, the oligonucleotide includes one or more nucleotide 5′ to the palindromic domain.

The palindromic domain may be linked, either directly or indirectly, to a Poly G domain. As used herein, the term “linked directly” refers to an oligonucleotide in which there is no intervening sequence between the palindromic domain and the Poly G domain. The term “linked indirectly” refers to an oligonucleotide in which the palindromic domain and the poly G domain are separated by a linker. In some embodiments the Poly G domain includes at least 3 and less than 8 consecutive Gs. When the palindromic domain is indirectly linked to the Poly G domain, the indirect linkage is comprised of a nucleotide sequence of 1-10 nucleotides or a non-nucleotide linker. A non-nucleotidic linker can be prepared using an additional spacer, such as tri- or tetra-ethylenglycol phosphate moiety (Durand, M. et al, Triple-helix formation by an oligonucleotide containing one (dA)12 and two (dT)12 sequences bridged by two hexaethylene glycol chains, Biochemistry (1992), 31(38), 9197-204, U.S. Pat. No. 5,658,738, and U.S. Pat. No. 5,668,265). Alternatively, the non-nucleotidic linker may be derived from ethanediol, propanediol, or from an abasic deoxyribose (dSpacer) unit (Fontanel, Marie Laurence et al., Sterical recognition by T4 polynucleotide kinase of non-nucleosidic moieties 5′-attached to oligonucleotides; Nucleic Acids Research (1994), 22(11), 2022-7) using standard phosphoramidite chemistry.

The modified A-class oligonucleotides contain stabilized internucleotide linkages, meaning they are are partially resistant to degradation (e.g., are stabilized). The oligonucleotides typically include at least 2 and less than 6 stabilized internucleotide linkages, but are not so limited. A stabilized oligonucleotide molecule shall mean an oligonucleotide that is relatively resistant to in vivo degradation (e.g. via an exo- or endo-nuclease). Nucleic acid stabilization can be accomplished via backbone modifications. Oligonucleotides having phosphorothioate linkages provide maximal activity and protect the oligonucleotide from degradation by intracellular exo- and endo-nucleases. Other modified oligonucleotides include phosphodiester modified nucleic acids, combinations of phosphodiester and phosphorothioate nucleic acid, methylphosphonate, methylphosphorothioate, phosphorodithioate, p-ethoxy, and combinations thereof.

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (e.g., Uhlmann, E. and Peyman, A., Chem. Rev. 90:544, 1990; Goodchild, J., Bioconjugate Chem. 1:165, 1990).

Other stabilized oligonucleotides include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acids which contain diol, such as tetraethyleneglycol or hexaethyleneglycol, at either or both termini have also been shown to be substantially resistant to nuclease degradation.

The stabilized internucleotide linkages typically occur in a part of the sequence outside the palindrome, such as the G-rich domain.

Some exemplary immunostimulatory oligonucleotides described by formula I are listed in table 1:

TABLE 1

| SEQ ID | |
|---|---|
| Number | Sequence 5′-3′ |
| 3 | T*C_G_A_C_G_T_C_G_T_G_G*G*G*G |
| 7 | T*C_G_T_C_G_A_C_G_T_G_G*G*G* |
| 8 | T*C_G_C_C_G_G_C_G_T_G_G*G*G*G |
| 9 | T*C_G_G_C_G_C_C_G_T_G_G*G*G*G |
| 10 | T*C_G_A_C_G_T_C_G_A_C_G_T_C_G_T_G_G*G*G*G |
| 11 | T*C_G_A_C_G_T_C_G_T_T_G_G*G*G*G |
| 12 | G*T_C_G_A_C_G_T_C_G_T_G_G*G*G*G |
| 13 | G*T*C_G_A_C_G_T_C_G_T_T_G_G*G*G*G |
| 14 | T*C_G_T_C_G_A_C_G_T_T_G_G*G*G*G |

Key
_ phosphodiester internucleotide bond
* phosphorothioate internucleotide bond

Those of ordinary skill in the art will be able to determine the sequence of other oligonucleotides belonging to this family of modified A-class oligonucleotides.

In another aspect of the invention the modified A-class oligonucleotides have a lipophilic moiety in place of the poly-G domain. A “lipophilic moiety” as used herein is a lipophilic group covalently attached to the 3′ end of the modified A-class oligonucleotide. The lipophilic group in general can be a cholesteryl, a modified cholesteryl, a cholesterol derivative, a reduced cholesterol, a substituted cholesterol, cholestan, C₁₋₆ alkyl chain, a bile acid, cholic acid, taurocholic acid, deoxycholate, oleyl litocholic acid, oleoyl cholenic acid, a glycolipid, a phospholipid, a sphingolipid, an isoprenoid, such as steroids, vitamins, such as vitamin E, saturated fatty acids, unsaturated fatty acids, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, or ibuprofen. In certain embodiments the lipophilic moiety is chosen from cholesteryl, palmityl, and fatty acyl. In one embodiment the lipohilic moiety is cholesteryl. It is believed that inclusion of one or more of such lipophilic moieties in the immunostimulatory oligonucleotides of the invention confers upon them yet additional stability against degradation by nucleases. Where there are two or more lipophilic moieties in a single immunostimulatory oligonucleotide of the invention, each lipophilic moiety can be selected independently of any other.

In one embodiment the lipophilic group is attached to a 2′-position of a nucleotide of the modified A-class oligonucleotide. A lipophilic group can alternatively or in addition be linked to the heterocyclic nucleobase of a nucleotide of the modified A-class oligonucleotide. The lipophilic moiety can be covalently linked to the modified A-class oligonucleotide via any suitable direct or indirect linkage. In one embodiment the linkage is direct and is an ester or an amide. In one embodiment the linkage is indirect and includes a spacer moiety, for example one or more abasic nucleotide residues, oligoethyleneglycol, such as triethyleneglycol (spacer 9) or hexaethylenegylcol (spacer 18), or an alkane-diol, such as butanediol.

The immunostimulatory oligonucleotides generally have a length in the range of between 4 and 100 nucleotides. In some embodiments the length is in the range of 4-40, 13-100, 13-40, 13-30, 15-50, or 15-30 nucleotides or any integer range therebetween. The oligonucleotides may be longer than 100 nucleotides in length. For instance they may be less than 120, 150 or 200 nucleotides in length. In some embodiments the immunostimulatory oligonucleotides are 15 or fewer nucleotides. In preferred embodiments, the immunostimulatory oligonucleotide is less than 16 nucleotides in length.

The terms “nucleic acid” and “oligonucleotide” are used interchangeably to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). As used herein, the terms “nucleic acid” and “oligonucleotide” refer to oligoribonucleotides as well as oligodeoxyribonucleotides. The terms “nucleic acid” and “oligonucleotide” shall also include polynucleosides (i.e., a polynucleotide minus the phosphate) and any other organic base containing polymer. Nucleic acid molecules can be obtained from existing nucleic acid sources (e.g., genomic or cDNA), but are preferably synthetic (e.g., produced by nucleic acid synthesis). The term oligonucleotide generally refers to a shorter molecule, i.e. 100 nucleotides or less in length.

The terms “nucleic acid” and “oligonucleotide” also encompass nucleic acids or oligonucleotides with substitutions or modifications, such as in the bases and/or sugars. For example, they include nucleic acids having backbone sugars that are covalently attached to low molecular weight organic groups other than a hydroxyl group at the 2′ position and other than a phosphate group or hydroxy group at the 5′ position. Thus modified nucleic acids may include a 2′-O-alkylated ribose group. In addition, modified nucleic acids may include sugars such as arabinose or 2′-fluoroarabinose instead of ribose. Thus the nucleic acids may be heterogeneous in backbone composition thereby containing any possible combination of polymer units linked together such as peptide-nucleic acids (which have an amino acid backbone with nucleic acid bases). Other examples are described in more detail below.

The immunostimulatory oligonucleotides of the instant invention can encompass various chemical modifications and substitutions, in comparison to natural RNA and DNA, involving a phosphodiester internucleoside bridge, a β-D-ribose unit and/or a natural nucleoside base (adenine, guanine, cytosine, thymine, uracil). Examples of chemical modifications are known to the skilled person and are described, for example, in Uhlmann E et al. (1990) Chem Rev 90:543; “Protocols for Oligonucleotides and Analogs” Synthesis and Properties & Synthesis and Analytical Techniques, S. Agrawal, Ed, Humana Press, Totowa, USA 1993; Crooke S T et al. (1996) Annu Rev Pharmacol Toxicol 36:107-129; and Hunziker J et al. (1995) Mod Synth Methods 7:331-417. An oligonucleotide according to the invention may have one or more modifications, wherein each modification is located at a particular phosphodiester internucleoside bridge and/or at a particular β-D-ribose unit and/or at a particular natural nucleoside base position in comparison to an oligonucleotide of the same sequence which is composed of natural DNA or RNA.

For example, the oligonucleotides may comprise one or more modifications and wherein each modification is independently selected from:
a) the replacement of a phosphodiester internucleoside bridge located at the 3′ and/or the 5′ end of a nucleoside by a modified internucleoside bridge,
b) the replacement of phosphodiester bridge located at the 3′ and/or the 5′ end of a nucleoside by a dephospho bridge,
c) the replacement of a sugar phosphate unit from the sugar phosphate backbone by another unit,
d) the replacement of a 13-D-ribose unit by a modified sugar unit, and
e) the replacement of a natural nucleoside base by a modified nucleoside base.

More detailed examples for the chemical modification of an oligonucleotide are as follows.

The oligonucleotides may include modified internucleotide linkages, such as those described in a or b above. These modified linkages may be partially resistant to degradation (e.g., are stabilized). A stabilized oligonucleotide molecule is an oligonucleotide that is relatively resistant to in vivo degradation (e.g. via an exo- or endo-nuclease) resulting form such modifications. Oligonucleotides having phosphorothioate linkages, in some embodiments, may provide maximal activity and protect the oligonucleotide from degradation by intracellular exo- and endo-nucleases. Typically A-class oligonucleotides have phosphorothioate or other stabilized bonds located at the 5′ and 3′ portions of the molecule. In some embodiments, the 3′ poly G domain is fully stabilized.

A phosphodiester internucleoside bridge located at the 3′ and/or the 5′ end of a nucleoside can be replaced by a modified internucleoside bridge, wherein the modified internucleoside bridge is for example selected from phosphorothioate, phosphorodithioate, NR¹R²-phosphoramidate, boranophosphate, α-hydroxybenzyl phosphonate, phosphate-(C₁-C₂₁)—O-alkyl ester, phosphate-[(C₆-C₁₂)aryl-(C₁-C₂₁)-β-alkyl]ester, (C₁-C₈)alkylphosphonate and/or (C₆-C₁₂)arylphosphonate bridges, (C₇-C₁₂)-α-hydroxymethykaryl (e.g., disclosed in WO 95/01363), wherein (C₈-C₁₂)aryl, (C₈-C₂₀)aryl and (C₆-C₁₄)aryl are optionally substituted by halogen, alkyl, alkoxy, nitro, cyano, and where R¹ and R² are, independently of each other, hydrogen, (C₁-C₁₈)-alkyl, (C₆-C₂₀)-aryl, (C₆-C₁₄)-aryl-(C₁-C₈)-alkyl, preferably hydrogen, (C₁-C₈)-alkyl, preferably (C₁-C₄)-alkyl and/or methoxyethyl, or R¹ and R² form, together with the nitrogen atom carrying them, a 5-6-membered heterocyclic ring which can additionally contain a further heteroatom from the group O, S and N.

The replacement of a phosphodiester bridge located at the 3′ and/or the 5′ end of a nucleoside by a dephospho bridge (dephospho bridges are described, for example, in Uhlmann E and Peyman A in “Methods in Molecular Biology”, Vol. 20, “Protocols for Oligonucleotides and Analogs”, S. Agrawal, Ed., Humana Press, Totowa 1993, Chapter 16, pp. 355 ff), wherein a dephospho bridge is for example selected from the dephospho bridges formacetal, 3′-thioformacetal, methylhydroxylamine, oxime, methylenedimethyl-hydrazo, dimethylenesulfone and/or silyl groups.

A sugar phosphate unit (i.e., a β-D-ribose and phosphodiester internucleoside bridge together forming a sugar phosphate unit) from the sugar phosphate backbone (i.e., a sugar phosphate backbone is composed of sugar phosphate units) can be replaced by another unit, wherein the other unit is for example suitable to build up a “morpholino-derivative” oligomer (as described, for example, in Stirchak E P et al. (1989) Nucleic Acids Res 17:6129-41), that is, e.g., the replacement by a morpholino-derivative unit; or to build up a polyamide nucleic acid (“PNA”; as described for example, in Nielsen P E et al. (1994) Bioconjug Chem 5:3-7), that is, e.g., the replacement by a PNA backbone unit, e.g., by 2-aminoethylglycine. The oligonucleotide may have other carbohydrate backbone modifications and replacements, such as peptide nucleic acids with phosphate groups (PHONA), locked nucleic acids (LNA), and oligonucleotides having backbone sections with alkyl linkers or amino linkers. The alkyl linker may be branched or unbranched, substituted or unsubstituted, and chirally pure or a racemic mixture.

A β-ribose unit or a β-D-2′-deoxyribose unit can be replaced by a modified sugar unit, wherein the modified sugar unit is for example selected from β-D-ribose, α-D-2′-deoxyribose, L-2′-deoxyribose, 2′-F-2′-deoxyribose, 2′-F-arabinose, 2′-O—(C₁-C₆)alkyl-ribose, preferably 2′-O—(C₁-C₆)alkyl-ribose is 2′-O-methylribose, 2′-O—(C₂-C₆)alkenyl-ribose, 2′[O—(C₁-C₆)alkyl-O—(C₁-C₆)alkyl]-ribose, 2′—NH₂-2′-deoxyribose, β-D-xylo-furanose, α-arabinofuranose, 2,4-dideoxy-β-D-erythro-hexo-pyranose, and carbocyclic (described, for example, in Froehler J (1992) Am Chem Soc 114:8320) and/or open-chain sugar analogs (described, for example, in Vandendriessche et al. (1993) Tetrahedron 49:7223) and/or bicyclosugar analogs (described, for example, in Tarkov M et al. (1993) Hely Chim Acta 76:481).

In some embodiments the sugar is 2′-O-methylribose, particularly for one or both nucleotides linked by a phosphodiester or phosphodiester-like internucleoside linkage.

Nucleic acids also include substituted purines and pyrimidines such as C-5 propyne pyrimidine and 7-deaza-7-substituted purine modified bases. Wagner R W et al. (1996) Nat Biotechnol 14:840-4. Purines and pyrimidines include but are not limited to adenine, cytosine, guanine, and thymine, and other naturally and non-naturally occurring nucleobases, substituted and unsubstituted aromatic moieties.

A modified base is any base which is chemically distinct from the naturally occurring bases typically found in DNA and RNA such as T, C, G, A, and U, but which share basic chemical structures with these naturally occurring bases. The modified nucleoside base may be, for example, selected from hypoxanthine, uracil, dihydrouracil, pseudouracil, 2-thiouracil, 4-thiouracil, 5-aminouracil, 5-(C₁-C₆)-alkyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 2,4-diamino-purine, 8-azapurine, a substituted 7-deazapurine, preferably 7-deaza-7-substituted and/or 7-deaza-8-substituted purine, 5-hydroxymethylcytosine, N4-alkylcytosine, e.g., N4-ethylcytosine, 5-hydroxydeoxycytidine, 5-hydroxymethyldeoxycytidine, N4-alkyldeoxycytidine, e.g., N4-ethyldeoxycytidine, 6-thiodeoxyguanosine, and deoxyribonucleosides of nitropyrrole, C5-propynylpyrimidine, and diaminopurine e.g., 2,6-diaminopurine, inosine, 5-methylcytosine, 2-aminopurine, 2-amino-6-chloropurine, hypoxanthine or other modifications of a natural nucleoside bases. This list is meant to be exemplary and is not to be interpreted to be limiting.

In the formulae described herein a set of modified bases is defined. For instance the letter Y is used to refer to a nucleotide wherein the nucleotide is a cytosine or a modified cytosine. A modified cytosine as used herein is a naturally occurring or non-naturally occurring pyrimidine base analog of cytosine which can replace this base without impairing the immunostimulatory activity of the oligonucleotide. Modified cytosines include but are not limited to 5-substituted cytosines (e.g. 5-methyl-cytosine, 5-fluoro-cytosine, 5-chloro-cytosine, 5-bromo-cytosine, 5-fluoro-cytosine 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g. N4-ethyl-cytosine), 5-aza-cytosine, 2-mercapto-cytosine, isocytosine, pseudo-isocytosine, cytosine analogs with condensed ring systems (e.g. N,N′-propylene cytosine or phenoxazine), and uracil and its derivatives (e.g. 5-fluoro-uracil, 5-bromo-uracil, 5-bromovinyl-uracil, 4-thio-uracil, 5-hydroxy-uracil, 5-propynyl-uracil). In certain embodiments, the modified cytosine residue corresponding to Y₁, Y₂, and Y₃ of formula I are each independently cytosine or 5-substituted cytosines such as 5-methyl-cytosine, 5-hydroxy-cytosine or 5-fluoro-cytosine. In another embodiment of the invention, the cytosine base is substituted by a universal base (e.g. 3-nitropyrrole, P-base), an aromatic ring system (e.g. fluorobenzene or difluorobenzene) or a hydrogen atom (dSpacer).

The letter R is used to refer to guanine or a modified guanine base. A modified guanine as used herein is a naturally occurring or non-naturally occurring purine base analog of guanine which can replace this base without impairing the immunostimulatory activity of the oligonucleotide. Modified guanines include but are not limited to 7-deaza-guanine, 7-deaza-7-substituted guanine (such as 7-deaza-7-(C2-C6)alkynylguanine), 7-deaza-8-substituted guanine, hypoxanthine, N2-substituted guanines (e.g. N2-methyl-guanine), 5-amino-3-methyl-3H,6H-thiazolo[4,5-d]pyrimidine-2,7-dione, 2,6-diaminopurine, 2-aminopurine, purine, indole, adenine, substituted adenines (e.g. N6-methyl-adenine, 8-oxo-adenine) 8-substituted guanine (e.g. 8-hydroxyguanine and 8-bromoguanine), and 6-thioguanine. In another embodiment of the invention, the guanine base is substituted by a universal base (e.g. 4-methyl-indole, 5-nitro-indole, and K-base), an aromatic ring system (e.g. benzimidazole or dichloro-benzimidazole, 1-methyl-1H-[1,2,4]triazole-3-carboxylic acid amide) or a hydrogen atom (dSpacer). In some embodiments the modified guanine corresponding to R₁, R₂ and R₃ of formula I are each independently guanine, inosine (I), 6-thio-guanine, or 7-deaza-guanine.

The oligonucleotides of the instant invention may include lipophilic nucleotide analogs. The modified A class oligonucleotides in some aspects comprise the sequence R₄Py-PuR₅, wherein R₄ and R₅ are each a lipophilic substituted nucleotide analog, wherein Py is a pyrimidine nucleotide and wherein Pu is a purine or an abasic residue. Preferred lipophilic nucleotide analogs are e.g. 5-chloro-uracil, 5-bromo-uracil, 5-iodo-uracil, 5-ethyl-uracil, 5-propyl-uracil, 2,4-difluoro-toluene, and 3-nitropyrrole.

For use in the instant invention, the oligonucleotides of the invention can be synthesized de novo using any of a number of procedures well known in the art. For example, the β-cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid. Res. 14:5399-5407, 1986; Garegg et al., Tet. Let. 27:4055-4058, 1986, Gaffney et al., Tet. Let. 29:2619-2622, 1988). These chemistries can be performed by a variety of automated nucleic acid synthesizers available in the market. These oligonucleotides are referred to as synthetic oligonucleotides. An isolated oligonucleotide generally refers to an oligonucleotide which is separated from components which it is normally associated with in nature. As an example, an isolated oligonucleotide may be one which is separated from a cell, from a nucleus, from mitochondria or from chromatin.

The internucleotide linkages in the oligonucleotide may be non-stabilized or stabilized linkages (against nucleases), preferably phosphodiester (non stabilized), a phosphorothioate (stabilized) or another charged backbone. If the internucleotide linkage at Y—R is a phosphorothioate, the chirality of this linkage may be random, or is preferably a phosphorothioate linkage of Rp configuration.

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (e.g., Uhlmann, E. and Peyman, A., Chem. Rev. 90:544, 1990; Goodchild, J., Bioconjugate Chem. 1:165, 1990).

Thus the modified A-class oligonucleotides are useful in some aspects of the invention for the treatment of a subject having or at risk of developing an infectious disease, cancer, allergy, asthma, autoimmune or inflammatory disease. As used herein, the terms treat, treated, or treating when used with respect to a disorder such as an infectious disease, cancer, allergy, asthma, autoimmune or inflammatory disease refers to a prophylactic treatment which increases the resistance of a subject to development of the disease (e.g., to infection with a pathogen) or, in other words, decreases the likelihood that the subject will develop the disease (e.g., become infected with the pathogen) as well as a treatment after the subject has developed the disease in order to fight the disease (e.g., reduce or eliminate the infection) or prevent the disease from becoming worse.

In one embodiment the modified A-class oligonucleotides are useful for treating a subject who has been previously unresponsive to conventional therapeutic treatments. Such a subject may be someone who has never responded to treatment or it may be someone who no longer response to a previously efficacious treatment. In other embodiments the subject has not been previously treated with these or other compounds.

A “subject” as used herein refers to a vertebrate animal. In various embodiments the subject is a human, a non-human primate, or other mammal. In certain embodiments the subject is a mouse, rat, guinea pig, rabbit, cat, dog, pig, sheep, goat, cow, or horse.

The modified A-class oligonucleotides of the invention can be administered alone or with an antigen. The antigen can be separate from or covalently linked to a modified A-class oligonucleotide of the invention. In one embodiment the composition of the invention does not itself include the antigen. In this embodiment the antigen can be administered to the subject either separately from the composition of the invention, or together with the composition of the invention. Administration that is separate includes separate in time, separate in location or route of administration, or separate both in time and in location or route of administration. When the composition of the invention and the antigen are administered separate in time, the antigen can be administered before or after the composition of the invention. In one embodiment the antigen is administered 48 hours to 4 weeks after administration of the composition of the invention. The method also contemplates the administration of one or more booster doses of antigen alone, composition alone, or antigen and composition, following an initial administration of antigen and composition.

It is also contemplated by the invention that a subject can be prepared for a future encounter with an unknown antigen by administering to the subject a composition of the invention, wherein the composition does not include an antigen. According to this embodiment the immune system of the subject is prepared to mount a more vigorous response to an antigen that is later encountered by the subject, for example through environmental or occupational exposure. Such method can be used, for example, for travellers, medical workers, and soldiers likely to be exposed to microbial agents.

The modified A class oligonucleotides of the invention may be administered alone or with other medicaments. In one aspect the invention provides a composition useful for the treatment of infection. The composition according to this aspect includes a modified A-class oligonucleotide of the invention and an anti-infection medicament.

A “subject having an infectious disease” is a subject that has a disorder arising from the invasion of the subject, superficially, locally, or systemically, by an infectious microorganism. The infectious microorganism can be a virus, bacterium, fungus, or parasite, as described above. As such, an infectious disease caused by the invasion of a virus is defined as a “viral disease”. A “subject at risk” of developing an infectious disease as used herein is a subject who has any risk of exposure to a microorganism, e.g. someone who is in contact with an infected subject or who is traveling to a place where a particular microorganism is found. For instance, a subject at risk may be a subject who is planning to travel to an area where a particular microorganism is found or it may even be any subject living in an area where a microorganism has been identified. A subject at risk of developing an infectious disease includes those subjects that have a general risk of exposure to a microorganism, e.g., influenza, but that don't have the active disease during the treatment of the invention as well as subjects that are considered to be at specific risk of developing an infectious disease because of medical or environmental factors, that expose them to a particular microorganism.

Infection medicaments include but are not limited to anti-bacterial agents, anti-viral agents, anti-fungal agents and anti-parasitic agents. Phrases such as “anti-infective agent”, “antibiotic”, “anti-bacterial agent”, “anti-viral agent”, “anti-fungal agent”, “anti-parasitic agent” and “parasiticide” have well-established meanings to those of ordinary skill in the art and are defined in standard medical texts. Briefly, anti-bacterial agents kill or inhibit bacteria, and include antibiotics as well as other synthetic or natural compounds having similar functions. Anti-viral agents can be isolated from natural sources or synthesized and are useful for killing or inhibiting viruses. Anti-fungal agents are used to treat superficial fungal infections as well as opportunistic and primary systemic fungal infections. Anti-parasite agents kill or inhibit parasites. Many antibiotics are low molecular weight molecules which are produced as secondary metabolites by cells, such as microorganisms. In general, antibiotics interfere with one or more functions or structures which are specific for the microorganism and which are not present in host cells.

One of the problems with anti-infective therapies is the side effects occurring in the host that is treated with the anti-infective agent. For instance, many anti-infectious agents can kill or inhibit a broad spectrum of microorganisms and are not specific for a particular type of species. Treatment with these types of anti-infectious agents results in the killing of the normal microbial flora living in the host, as well as the infectious microorganism. The loss of the microbial flora can lead to disease complications and predispose the host to infection by other pathogens, since the microbial flora compete with and function as barriers to infectious pathogens. Other side effects may arise as a result of specific or non-specific effects of these chemical entities on non-microbial cells or tissues of the host.

Another problem with widespread use of anti-infectants is the development of antibiotic-resistant strains of microorganisms. Already, vancomycin-resistant enterococci, penicillin-resistant pneumococci, multi-resistant S. aureus, and multi-resistant tuberculosis strains have developed and are becoming major clinical problems. Widespread use of anti-infectants will likely produce many antibiotic-resistant strains of bacteria. As a result, new anti-infective strategies will be required to combat these microorganisms.

Antibacterial antibiotics which are effective for killing or inhibiting a wide range of bacteria are referred to as broad-spectrum antibiotics. Other types of antibacterial antibiotics are predominantly effective against the bacteria of the class gram-positive or gram-negative. These types of antibiotics are referred to as narrow-spectrum antibiotics. Other antibiotics which are effective against a single organism or disease and not against other types of bacteria, are referred to as limited-spectrum antibiotics.

Anti-bacterial agents are sometimes classified based on their primary mode of action. In general, anti-bacterial agents are cell wall synthesis inhibitors, cell membrane inhibitors, protein synthesis inhibitors, nucleic acid synthesis or functional inhibitors, and competitive inhibitors. Cell wall synthesis inhibitors inhibit a step in the process of cell wall synthesis, and in general in the synthesis of bacterial peptidoglycan. Cell wall synthesis inhibitors include β-lactam antibiotics, natural penicillins, semi-synthetic penicillins, ampicillin, clavulanic acid, cephalolsporins, and bacitracin.

The β-lactams are antibiotics containing a four-membered β-lactam ring which inhibits the last step of peptidoglycan synthesis. β-lactam antibiotics can be synthesized or natural. The β-lactam antibiotics produced by penicillium are the natural penicillins, such as penicillin G or penicillin V. These are produced by fermentation of Penicillium chrysogenum. The natural penicillins have a narrow spectrum of activity and are generally effective against Streptococcus, Gonococcus, and Staphylococcus. Other types of natural penicillins, which are also effective against gram-positive bacteria, include penicillins F, X, K, and O.

Semi-synthetic penicillins are generally modifications of the molecule 6-aminopenicillanic acid produced by a mold. The 6-aminopenicillanic acid can be modified by addition of side chains which produce penicillins having broader spectrums of activity than natural penicillins or various other advantageous properties. Some types of semi-synthetic penicillins have broad spectrums against gram-positive and gram-negative bacteria, but are inactivated by penicillinase. These semi-synthetic penicillins include ampicillin, carbenicillin, oxacillin, azlocillin, mezlocillin, and piperacillin. Other types of semi-synthetic penicillins have narrower activities against gram-positive bacteria, but have developed properties such that they are not inactivated by penicillinase. These include, for instance, methicillin, dicloxacillin, and nafcillin. Some of the broad spectrum semi-synthetic penicillins can be used in combination with β-lactamase inhibitors, such as clavulanic acids and sulbactam. The β-lactamase inhibitors do not have anti-microbial action but they function to inhibit penicillinase, thus protecting the semi-synthetic penicillin from degradation.

Another type of β-lactam antibiotic is the cephalolsporins. They are sensitive to degradation by bacterial β-lactamases, and thus, are not always effective alone. Cephalolsporins, however, are resistant to penicillinase. They are effective against a variety of gram-positive and gram-negative bacteria. Cephalolsporins include, but are not limited to, cephalothin, cephapirin, cephalexin, cefamandole, cefaclor, cefazolin, cefuroxine, cefoxitin, cefotaxime, cefsulodin, cefetamet, cefixime, ceftriaxone, cefoperazone, ceftazidine, and moxalactam.

Bacitracin is another class of antibiotics which inhibit cell wall synthesis, by inhibiting the release of muropeptide subunits or peptidoglycan from the molecule that delivers the subunit to the outside of the membrane. Although bacitracin is effective against gram-positive bacteria, its use is limited in general to topical administration because of its high toxicity.

Carbapenems are another broad-spectrum β-lactam antibiotic, which is capable of inhibiting cell wall synthesis. Examples of carbapenems include, but are not limited to, imipenems. Monobactams are also broad-spectrum β-lactam antibiotics, and include, eurtreonam. An antibiotic produced by Streptomyces, vancomycin, is also effective against gram-positive bacteria by inhibiting cell membrane synthesis.

Another class of anti-bacterial agents is the anti-bacterial agents that are cell membrane inhibitors. These compounds disorganize the structure or inhibit the function of bacterial membranes. One problem with anti-bacterial agents that are cell membrane inhibitors is that they can produce effects in eukaryotic cells as well as bacteria because of the similarities in phospholipids in bacterial and eukaryotic membranes. Thus these compounds are rarely specific enough to permit these compounds to be used systemically and prevent the use of high doses for local administration.

One clinically useful cell membrane inhibitor is Polymyxin. Polymyxins interfere with membrane function by binding to membrane phospholipids. Polymyxin is effective mainly against Gram-negative bacteria and is generally used in severe Pseudomonas infections or Pseudomonas infections that are resistant to less toxic antibiotics. The severe side effects associated with systemic administration of this compound include damage to the kidney and other organs.

Other cell membrane inhibitors include Amphotericin B and Nystatin which are anti-fungal agents used predominantly in the treatment of systemic fungal infections and Candida yeast infections. Imidazoles are another class of antibiotic that is a cell membrane inhibitor. Imidazoles are used as anti-bacterial agents as well as anti-fungal agents, e.g., used for treatment of yeast infections, dermatophytic infections, and systemic fungal infections. Imidazoles include but are not limited to clotrimazole, miconazole, ketoconazole, itraconazole, and fluconazole.

Many anti-bacterial agents are protein synthesis inhibitors. These compounds prevent bacteria from synthesizing structural proteins and enzymes and thus cause inhibition of bacterial cell growth or function or cell death. In general these compounds interfere with the processes of transcription or translation. Anti-bacterial agents that block transcription include but are not limited to Rifampins and Ethambutol. Rifampins, which inhibit the enzyme RNA polymerase, have a broad spectrum activity and are effective against gram-positive and gram-negative bacteria as well as Mycobacterium tuberculosis. Ethambutol is effective against Mycobacterium tuberculosis.

Anti-bacterial agents which block translation interfere with bacterial ribosomes to prevent mRNA from being translated into proteins. In general this class of compounds includes but is not limited to tetracyclines, chloramphenicol, the macrolides (e.g., erythromycin) and the aminoglycosides (e.g., streptomycin).

The aminoglycosides are a class of antibiotics which are produced by the bacterium Streptomyces, such as, for instance streptomycin, kanamycin, tobramycin, amikacin, and gentamicin. Aminoglycosides have been used against a wide variety of bacterial infections caused by Gram-positive and Gram-negative bacteria. Streptomycin has been used extensively as a primary drug in the treatment of tuberculosis. Gentamicin is used against many strains of Gram-positive and Gram-negative bacteria, including Pseudomonas infections, especially in combination with Tobramycin. Kanamycin is used against many Gram-positive bacteria, including penicillin-resistant Staphylococci. One side effect of aminoglycosides that has limited their use clinically is that at dosages which are essential for efficacy, prolonged use has been shown to impair kidney function and cause damage to the auditory nerves leading to deafness.

Another type of translation inhibitor anti-bacterial agent is the tetracyclines. The tetracyclines are a class of antibiotics that are broad-spectrum and are effective against a variety of gram-positive and gram-negative bacteria. Examples of tetracyclines include tetracycline, minocycline, doxycycline, and chlortetracycline. They are important for the treatment of many types of bacteria but are particularly important in the treatment of Lyme disease. As a result of their low toxicity and minimal direct side effects, the tetracyclines have been overused and misused by the medical community, leading to problems. For instance, their overuse has led to widespread development of resistance.

Anti-bacterial agents such as the macrolides bind reversibly to the 50 S ribosomal subunit and inhibit elongation of the protein by peptidyl transferase or prevent the release of uncharged tRNA from the bacterial ribosome or both. These compounds include erythromycin, roxithromycin, clarithromycin, oleandomycin, and azithromycin. Erythromycin is active against most Gram-positive bacteria, Neisseria, Legionella and Haemophilus, but not against the Enterobacteriaceae. Lincomycin and clindamycin, which block peptide bond formation during protein synthesis, are used against gram-positive bacteria.

Another type of translation inhibitor is chloramphenicol. Chloramphenicol binds the 70 S ribosome inhibiting the bacterial enzyme peptidyl transferase thereby preventing the growth of the polypeptide chain during protein synthesis. One serious side effect associated with chloramphenicol is aplastic anemia. Aplastic anemia develops at doses of chloramphenicol which are effective for treating bacteria in a small proportion (1/50,000) of patients. Chloramphenicol which was once a highly prescribed antibiotic is now seldom uses as a result of the deaths from anemia. Because of its effectiveness it is still used in life-threatening situations (e.g., typhoid fever).

Some anti-bacterial agents disrupt nucleic acid synthesis or function, e.g., bind to DNA or RNA so that their messages cannot be read. These include but are not limited to quinolones and co-trimoxazole, both synthetic chemicals and rifamycins, a natural or semi-synthetic chemical. The quinolones block bacterial DNA replication by inhibiting the DNA gyrase, the enzyme needed by bacteria to produce their circular DNA. They are broad spectrum and examples include norfloxacin, ciprofloxacin, enoxacin, nalidixic acid and temafloxacin. Nalidixic acid is a bactericidal agent that binds to the DNA gyrase enzyme (topoisomerase) which is essential for DNA replication and allows supercoils to be relaxed and reformed, inhibiting DNA gyrase activity. The main use of nalidixic acid is in treatment of lower urinary tract infections (UTI) because it is effective against several types of Gram-negative bacteria such as E. coli, Enterobacter aerogenes, K. pneumoniae and Proteus species which are common causes of UTI. Co-trimoxazole is a combination of sulfamethoxazole and trimethoprim, which blocks the bacterial synthesis of folic acid needed to make DNA nucleotides. Rifampicin is a derivative of rifamycin that is active against Gram-positive bacteria (including Mycobacterium tuberculosis and meningitis caused by Neisseria meningitidis) and some Gram-negative bacteria. Rifampicin binds to the beta subunit of the polymerase and blocks the addition of the first nucleotide which is necessary to activate the polymerase, thereby blocking mRNA synthesis.

Another class of anti-bacterial agents is compounds that function as competitive inhibitors of bacterial enzymes. The competitive inhibitors are mostly all structurally similar to a bacterial growth factor and compete for binding but do not perform the metabolic function in the cell. These compounds include sulfonamides and chemically modified forms of sulfanilamide which have even higher and broader antibacterial activity. The sulfonamides (e.g., gantrisin and trimethoprim) are useful for the treatment of Streptococcus pneumoniae, beta-hemolytic streptococci and E. coli, and have been used in the treatment of uncomplicated UTI caused by E. coli, and in the treatment of meningococcal meningitis.

Anti-viral agents are compounds which prevent infection of cells by viruses or replication of the virus within the cell. There are many fewer anti-viral drugs than antibacterial drugs because the process of viral replication is so closely related to DNA replication within the host cell, that non-specific anti-viral agents would often be toxic to the host. There are several stages within the process of viral infection which can be blocked or inhibited by anti-viral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleoside analogs), maturation of new virus proteins (e.g. protease inhibitors), and budding and release of the virus.

Another category of anti-viral agents are nucleoside analogs. Nucleoside analogs are synthetic compounds which are similar to nucleosides, but which have an incomplete or abnormal deoxyribose or ribose group. Once the nucleoside analogs are in the cell, they are phosphorylated, producing the triphosphate form which competes with normal nucleotides for incorporation into the viral DNA or RNA. Once the triphosphate form of the nucleoside analogue is incorporated into the growing nucleic acid chain, it causes irreversible association with the viral polymerase and thus chain termination. Nucleoside analogs include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, and zidovudine (azidothymidine).

Another class of anti-viral agents includes cytokines such as interferons. The interferons are cytokines which are secreted by virus-infected cells as well as immune cells. The interferons function by binding to specific receptors on cells adjacent to the infected cells, causing the change in the cell which protects it from infection by the virus. α and β-interferon also induce the expression of Class I and Class II MHC molecules on the surface of infected cells, resulting in increased antigen presentation for host immune cell recognition. α and β-interferons are available as recombinant forms and have been used for the treatment of chronic hepatitis B and C infection. At the dosages which are effective for anti-viral therapy, interferons have severe side effects such as fever, malaise and weight loss.

Immunoglobulin therapy is used for the prevention of viral infection. Immunoglobulin therapy for viral infections is different from bacterial infections, because rather than being antigen-specific, the immunoglobulin therapy functions by binding to extracellular virions and preventing them from attaching to and entering cells which are susceptible to the viral infection. The therapy is useful for the prevention of viral infection for the period of time that the antibodies are present in the host. In general there are two types of immunoglobulin therapies, normal immune globulin therapy and hyper-immune globulin therapy. Normal immune globulin therapy utilizes a antibody product which is prepared from the serum of normal blood donors and pooled. This pooled product contains low titers of antibody to a wide range of human viruses, such as hepatitis A, parvovirus, enterovirus (especially in neonates). Hyper-immune globulin therapy utilizes antibodies which are prepared from the serum of individuals who have high titers of an antibody to a particular virus. Those antibodies are then used against a specific virus. Examples of hyper-immune globulins include zoster immune globulin (useful for the prevention of varicella in immunocompromised children and neonates), human rabies immune globulin (useful in the post-exposure prophylaxis of a subject bitten by a rabid animal), hepatitis B immune globulin (useful in the prevention of hepatitis B virus, especially in a subject exposed to the virus), and RSV immune globulin (useful in the treatment of respiratory syncitial virus infections).

Anti-viral agents or medicaments known in the art include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscamet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

Anti-fungal agents are useful for the treatment and prevention of infective fungi. Anti-fungal agents are sometimes classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, imidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g., chitinase) or immunosuppression (501 cream).

Parasiticides are agents that kill parasites directly. Such compounds are known in the art and are generally commercially available. Examples of parasiticides useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide.

The modified A-class oligonucleotides are also useful for treating and preventing autoimmune disease. Autoimmune disease is a class of diseases in which a subject's own antibodies react with host tissue or in which immune effector T cells are autoreactive to endogenous self peptides and cause destruction of tissue. Thus an immune response is mounted against a subject's own antigens, referred to as self antigens. Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus.

A “self-antigen” as used herein refers to an antigen of a normal host tissue. Normal host tissue does not include cancer cells. Thus an immune response mounted against a self-antigen, in the context of an autoimmune disease, is an undesirable immune response and contributes to destruction and damage of normal tissue, whereas an immune response mounted against a cancer antigen is a desirable immune response and contributes to the destruction of the tumor or cancer. Thus, in some aspects of the invention aimed at treating autoimmune disorders it is not recommended that the oligonucleotide be administered with self antigens, particularly those that are the targets of the autoimmune disorder.

In other instances, the modified A-class oligonucleotides may be delivered with low doses of self-antigens. A number of animal studies have demonstrated that mucosal administration of low doses of antigen can result in a state of immune hyporesponsiveness or “tolerance.” The active mechanism appears to be a cytokine-mediated immune deviation away from a Th1 towards a predominantly Th2 and Th3 (i.e., TGF-□ dominated) response. The active suppression with low dose antigen delivery can also suppress an unrelated immune response (bystander suppression) which is of considerable interest in the therapy of autoimmune diseases, for example, rheumatoid arthritis and SLE. Bystander suppression involves the secretion of Th1-counter-regulatory, suppressor cytokines in the local environment where proinflammatory and Th1 cytokines are released in either an antigen-specific or antigen-nonspecific manner. “Tolerance” as used herein is used to refer to this phenomenon. Indeed, oral tolerance has been effective in the treatment of a number of autoimmune diseases in animals including: experimental autoimmune encephalomyelitis (EAE), experimental autoimmune myasthenia gravis, collagen-induced arthritis (CIA), and insulin-dependent diabetes mellitus. In these models, the prevention and suppression of autoimmune disease is associated with a shift in antigen-specific humoral and cellular responses from a Th1 to Th2/Th3 response.

The compositions and methods of the invention can be used alone or in conjunction with other agents and methods useful for the treatment of cancer. In one aspect the invention provides a method of treating a subject having a cancer. The method according to this aspect of the invention includes the step of administering to a subject having a cancer an effective amount of a composition of the invention to treat the subject.

A subject having a cancer is a subject that has detectable cancerous cells. The cancer may be a malignant or non-malignant cancer. “Cancer” as used herein refers to an uncontrolled growth of cells which interferes with the normal functioning of the bodily organs and systems. Cancers which migrate from their original location and seed vital organs can eventually lead to the death of the subject through the functional deterioration of the affected organs. Hemopoietic cancers, such as leukemia, are able to outcompete the normal hemopoietic compartments in a subject, thereby leading to hemopoietic failure (in the form of anemia, thrombocytopenia and neutropenia) ultimately causing death. A “subject at risk of developing cancer” is a subject for whom the likelihood of developing cancer is higher than normal due to factors such as a family history of cancer, exposure to carcinogens, etc.

A metastasis is a region of cancer cells, distinct from the primary tumor location, resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Cancers include, but are not limited to, basal cell carcinoma, biliary tract cancer; bladder cancer; bone cancer; brain and central nervous system (CNS) cancer; breast cancer; cervical cancer; choriocarcinoma; colon and rectum cancer; connective tissue cancer; cancer of the digestive system; endometrial cancer; esophageal cancer; eye cancer; cancer of the head and neck; intra-epithelial neoplasm; kidney cancer; larynx cancer; leukemia; liver cancer; lung cancer (e.g. small cell and non-small cell); lymphoma including Hodgkin's and Non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; sarcoma; skin cancer; stomach cancer; testicular cancer; thyroid cancer; uterine cancer; cancer of the urinary system, as well as other carcinomas, adenocarcinomas, and sarcomas.

The immunostimulatory composition of the invention may also be administered in conjunction with an anti-cancer therapy. Anti-cancer therapies include cancer medicaments, radiation, and surgical procedures. As used herein, a “cancer medicament” refers to an agent which is administered to a subject for the purpose of treating a cancer. As used herein, “treating cancer” includes preventing the development of a cancer, reducing the symptoms of cancer, and/or inhibiting the growth of an established cancer. In other aspects, the cancer medicament is administered to a subject at risk of developing a cancer for the purpose of reducing the risk of developing the cancer. Various types of medicaments for the treatment of cancer are described herein. For the purpose of this specification, cancer medicaments are classified as chemotherapeutic agents, immunotherapeutic agents, cancer vaccines, hormone therapy, and biological response modifiers.

The chemotherapeutic agent may be selected from the group consisting of methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MMI270, BAY 12-9566, RAS farnesyl transferase inhibitor, farnesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, IS1641, ODN 698, TA 2516/Marmistat, BB2516/Marmistat, CDP 845, D2163, PD183805, DX8951f, Lemonal DP 2202, FK 317, Picibanil/OK-432, AD 32Nalrubicin, Metastron/strontium derivative, Temodal/Temozolomide, Evacet/liposomal doxorubicin, Yewtaxan/Paclitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT (Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′ deoxycoformycin), Semustine (methyl-CCNU), Teniposide (VM-26) and Vindesine sulfate, but it is not so limited.

The immunotherapeutic agent may be selected from the group consisting of 3622W94, 4B5, ANA Ab, anti-FLK-2, anti-VEGF, ATRAGEN, AVASTIN (bevacizumab; Genentech), BABS, BEC2, BEXXAR (tositumomab; GlaxoSmithKline), C225, CAMPATH (alemtuzumab; Genzyme Corp.), CEACIDE, CMA 676, EMD-72000, ERBITUX (cetuximab; ImClone Systems, Inc.), Gliomab-H, GNI-250, HERCEPTIN (trastuzumab; Genentech), IDEC-Y2B8, ImmuRAIT-CEA, ior c5, ior egf.r3, ior t6, LDP-03, LymphoCide, MDX-11, MDX-22, MDX-210, MDX-220, MDX-260, MDX-447, MELIMMUNE-1, MELIMMUNE-2, Monopharm-C, NovoMAb-G2, Oncolym, OV103, Ovarex, Panorex, Pretarget, Quadramet, Ributaxin, RITUXAN (rituximab; Genentech), SMART 1D10 Ab, SMART ABL 364 Ab, SMART M195, TNT, and ZENAPAX (daclizumab; Roche), but it is not so limited.

The cancer vaccine may be selected from the group consisting of EGF, Anti-idiotypic cancer vaccines, Gp75 antigen, GMK melanoma vaccine, MGV ganglioside conjugate vaccine, Her2/neu, Ovarex, M-Vax, O-Vax, L-Vax, STn-KHL theratope, BLP25 (MUC-1), liposomal idiotypic vaccine, Melacine, peptide antigen vaccines, toxin/antigen vaccines, MVA-based vaccine, PACIS, BCG vacine, TA-HPV, TA-CIN, DISC-virus and ImmuCyst/TheraCys, but it is not so limited.

The compositions and methods of the invention can be used alone or in conjunction with other agents and methods useful for the treatment of allergy. In one aspect the invention provides a method of treating a subject having an allergic condition. The method according to this aspect of the invention includes the step of administering to a subject having an allergic condition an effective amount of a composition of the invention to treat the subject.

In one aspect the invention provides a method of treating a subject having an allergic condition. The method according to this aspect of the invention includes the step of administering to a subject having an allergic condition an effective amount of the composition of the invention and an anti-allergy therapy to treat the subject.

In one aspect the invention provides a use of a modified A-class oligonucleotide of the invention for the preparation of a medicament for treating an allergic condition in a subject.

In one aspect the invention provides a composition useful for the treatment of an allergic condition. The composition according to this aspect includes a modified A-class oligonucleotide of the invention and an allergy medicament.

A “subject having an allergic condition” shall refer to a subject that is currently experiencing or has previously experienced an allergic reaction in response to an allergen. An “allergic condition” or “allergy” refers to acquired hypersensitivity to a substance (allergen). Allergic conditions include but are not limited to eczema, allergic rhinitis or coryza, hay fever, allergic conjunctivitis, bronchial asthma, urticaria (hives) and food allergies, other atopic conditions including atopic dermatitis; anaphylaxis; drug allergy; and angioedema.

Allergy is typically an episodic condition associated with the production of antibodies from a particular class of immunoglobulin, IgE, against allergens. The development of an IgE-mediated response to common aeroallergens is also a factor which indicates predisposition towards the development of asthma. If an allergen encounters a specific IgE which is bound to an IgE Fc receptor (Fc□R) on the surface of a basophil (circulating in the blood) or mast cell (dispersed throughout solid tissue), the cell becomes activated, resulting in the production and release of mediators such as histamine, serotonin, and lipid mediators.

An allergic reaction occurs when tissue-sensitizing immunoglobulin of the IgE type reacts with foreign allergen. The IgE antibody is bound to mast cells and/or basophils, and these specialized cells release chemical mediators (vasoactive amines) of the allergic reaction when stimulated to do so by allergens bridging the ends of the antibody molecule. Histamine, platelet activating factor, arachidonic acid metabolites, and serotonin are among the best known mediators of allergic reactions in man. Histamine and the other vasoactive amines are normally stored in mast cells and basophil leukocytes. The mast cells are dispersed throughout animal tissue and the basophils circulate within the vascular system. These cells manufacture and store histamine within the cell unless the specialized sequence of events involving IgE binding occurs to trigger its release.

Symptoms of an allergic reaction vary, depending on the location within the body where the IgE reacts with the antigen. If the reaction occurs along the respiratory epithelium, the symptoms generally are sneezing, coughing and asthmatic reactions. If the interaction occurs in the digestive tract, as in the case of food allergies, abdominal pain and diarrhea are common. Systemic allergic reactions, for example following a bee sting or administration of penicillin to an allergic subject, can be severe and often life-threatening.

Allergy is associated with a Th2-type of immune response, which is characterized at least in part by Th2 cytokines IL-4 and IL-5, as well as antibody isotype switching to IgE. Th1 and Th2 immune responses are mutually counter-regulatory, so that skewing of the immune response toward a Th1-type of immune response can prevent or ameliorate a Th2-type of immune response, including allergy. The modified A-class oligonucleotides of the invention are therefore useful by themselves to treat a subject having an allergic condition because the modified oligonucleotides can skew the immune response toward a Th1-type of immune response. Alternatively or in addition, the modified A-class oligonucleotides of the invention can be used in combination with an allergen to treat a subject having an allergic condition.

The immunostimulatory composition of the invention may also be administered in conjunction with an anti-allergy therapy. Conventional methods for treating or preventing allergy have involved the use of allergy medicaments or desensitization therapies. Some evolving therapies for treating or preventing allergy include the use of neutralizing anti-IgE antibodies. Anti-histamines and other drugs which block the effects of chemical mediators of the allergic reaction help to regulate the severity of the allergic symptoms but do not prevent the allergic reaction and have no effect on subsequent allergic responses. Desensitization therapies are performed by giving small doses of an allergen, usually by injection under the skin, in order to induce an IgG-type response against the allergen. The presence of IgG antibody helps to neutralize the production of mediators resulting from the induction of IgE antibodies, it is believed. Initially, the subject is treated with a very low dose of the allergen to avoid inducing a severe reaction and the dose is slowly increased. This type of therapy is dangerous because the subject is actually administered the compounds which cause the allergic response and severe allergic reactions can result.

Allergy medicaments include, but are not limited to, anti-histamines, corticosteroids, and prostaglandin inducers. Anti-histamines are compounds which counteract histamine released by mast cells or basophils. These compounds are well known in the art and commonly used for the treatment of allergy. Anti-histamines include, but are not limited to, acrivastine, astemizole, azatadine, azelastine, betatastine, brompheniramine, buclizine, cetirizine, cetirizine analogs, chlorpheniramine, clemastine, CS 560, cyproheptadine, desloratadine, dexchlorpheniramine, ebastine, epinastine, fexofenadine, HSR 609, hydroxyzine, levocabastine, loratidine, methscopolamine, mizolastine, norastemizole, phenindamine, promethazine, pyrilamine, terfenadine, and tranilast.

Corticosteroids include, but are not limited to, methylprednisolone, prednisolone, prednisone, beclomethasone, budesonide, dexamethasone, flunisolide, fluticasone propionate, and triamcinolone. Although dexamethasone is a corticosteroid having anti-inflammatory action, it is not regularly used for the treatment of allergy or asthma in an inhaled form because it is highly absorbed and it has long-term suppressive side effects at an effective dose. Dexamethasone, however, can be used according to the invention for treating allergy or asthma because when administered in combination with a composition of the invention it can be administered at a low dose to reduce the side effects. Some of the side effects associated with corticosteroid use include cough, dysphonia, oral thrush (candidiasis), and in higher doses, systemic effects, such as adrenal suppression, glucose intolerance, osteoporosis, aseptic necrosis of bone, cataract formation, growth suppression, hypertension, muscle weakness, skin thinning, and easy bruising. Barnes & Peterson (1993) Am Rev Respir Dis 148:S1-S26; and Kamada A K et al. (1996) Am J Respir Crit Care Med 153:1739-48.

The compositions and methods of the invention can be used alone or in conjunction with other agents and methods useful for the treatment of asthma. In one aspect the invention provides a method of treating a subject having asthma. The method according to this aspect of the invention includes the step of administering to a subject having asthma an effective amount of a composition of the invention to treat the subject.

In one aspect the invention provides a method of treating a subject having asthma. The method according to this aspect of the invention includes the step of administering to a subject having asthma an effective amount of the composition of the invention and an anti-asthma therapy to treat the subject.

In one aspect the invention provides a use of a modified A-class oligonucleotide of the invention for the preparation of a medicament for treating asthma in a subject.

In one aspect the invention provides a composition useful for the treatment of asthma. The composition according to this aspect includes a modified A-class oligonucleotide of the invention and an asthma medicament.

“Asthma” as used herein refers to a disorder of the respiratory system characterized by inflammation and narrowing of the airways, and increased reactivity of the airways to inhaled agents. Asthma is frequently, although not exclusively, associated with an atopic or allergic condition. Symptoms of asthma include recurrent episodes of wheezing, breathlessness, chest tightness, and coughing, resulting from airflow obstruction. Airway inflammation associated with asthma can be detected through observation of a number of physiological changes, such as, denudation of airway epithelium, collagen deposition beneath basement membrane, edema, mast cell activation, inflammatory cell infiltration, including neutrophils, eosinophils, and lymphocytes. As a result of the airway inflammation, asthma patients often experience airway hyper-responsiveness, airflow limitation, respiratory symptoms, and disease chronicity. Airflow limitations include acute bronchoconstriction, airway edema, mucous plug formation, and airway remodeling, features which often lead to bronchial obstruction. In some cases of asthma, sub-basement membrane fibrosis may occur, leading to persistent abnormalities in lung function.

Research over the past several years has revealed that asthma likely results from complex interactions among inflammatory cells, mediators, and other cells and tissues resident in the airways. Mast cells, eosinophils, epithelial cells, macrophage, and activated T cells all play an important role in the inflammatory process associated with asthma. Djukanovic R et al. (1990) Am Rev Respir Dis 142:434-457. It is believed that these cells can influence airway function through secretion of preformed and newly synthesized mediators which can act directly or indirectly on the local tissue. It has also been recognized that subpopulations of T lymphocytes (Th2) play an important role in regulating allergic inflammation in the airway by releasing selective cytokines and establishing disease chronicity. Robinson D S et al. (1992) N Engl J Med 326:298-304.

Asthma is a complex disorder which arises at different stages in development and can be classified based on the degree of symptoms as acute, subacute, or chronic. An acute inflammatory response is associated with an early recruitment of cells into the airway. The subacute inflammatory response involves the recruitment of cells as well as the activation of resident cells causing a more persistent pattern of inflammation. Chronic inflammatory response is characterized by a persistent level of cell damage and an ongoing repair process, which may result in permanent abnormalities in the airway.

A “subject having asthma” is a subject that has a disorder of the respiratory system characterized by inflammation and narrowing of the airways and increased reactivity of the airways to inhaled agents. Factors associated with initiation of asthma include, but are not limited to, allergens, cold temperature, exercise, viral infections, and SO₂.

As mentioned above, asthma may be associated with a Th2-type of immune response, which is characterized at least in part by Th2 cytokines IL-4 and IL-5, as well as antibody isotype switching to IgE. Th1 and Th2 immune responses are mutually counter-regulatory, so that skewing of the immune response toward a Th1-type of immune response can prevent or ameliorate a Th2-type of immune response, including allergy. The modified oligonucleotide analogs of the invention are therefore useful by themselves to treat a subject having asthma because the analogs can skew the immune response toward a Th1-type of immune response. Alternatively or in addition, the modified oligonucleotide analogs of the invention can be used in combination with an allergen to treat a subject having asthma.

The immunostimulatory composition of the invention may also be administered in conjunction with an asthma therapy. Conventional methods for treating or preventing asthma have involved the use of anti-allergy therapies (described above) and a number of other agents, including inhaled agents.

Medications for the treatment of asthma are generally separated into two categories, quick-relief medications and long-term control medications. Asthma patients take the long-term control medications on a daily basis to achieve and maintain control of persistent asthma. Long-term control medications include anti-inflammatory agents such as corticosteroids, chromolyn sodium and nedocromil; long-acting bronchodilators, such as long-acting β₂-agonists and methylxanthines; and leukotriene modifiers. The quick-relief medications include short-acting β₂ agonists, anti-cholinergics, and systemic corticosteroids. There are many side effects associated with each of these drugs and none of the drugs alone or in combination is capable of preventing or completely treating asthma.

Asthma medicaments include, but are not limited, PDE-4 inhibitors, bronchodilator/beta-2 agonists, K+ channel openers, VLA-4 antagonists, neurokin antagonists, thromboxane A2 (TXA2) synthesis inhibitors, xanthines, arachidonic acid antagonists, 5 lipoxygenase inhibitors, TXA2 receptor antagonists, TXA2 antagonists, inhibitor of 5-lipox activation proteins, and protease inhibitors.

Bronchodilator/□₂ agonists are a class of compounds which cause bronchodilation or smooth muscle relaxation. Bronchodilator/□₂ agonists include, but are not limited to, salmeterol, salbutamol, albuterol, terbutaline, D2522/formoterol, fenoterol, bitolterol, pirbuerol methylxanthines and orciprenaline. Long-acting β₂ agonists and bronchodilators are compounds which are used for long-term prevention of symptoms in addition to the anti-inflammatory therapies. Long-acting β₂ agonists include, but are not limited to, salmeterol and albuterol. These compounds are usually used in combination with corticosteroids and generally are not used without any inflammatory therapy. They have been associated with side effects such as tachycardia, skeletal muscle tremor, hypokalemia, and prolongation of QTc interval in overdose.

Methylxanthines, including for instance theophylline, have been used for long-term control and prevention of symptoms. These compounds cause bronchodilation resulting from phosphodiesterase inhibition and likely adenosine antagonism. Dose-related acute toxicities are a particular problem with these types of compounds. As a result, routine serum concentration must be monitored in order to account for the toxicity and narrow therapeutic range arising from individual differences in metabolic clearance. Side effects include tachycardia, tachyarrhythmias, nausea and vomiting, central nervous system stimulation, headache, seizures, hematemesis, hyperglycemia and hypokalemia. Short-acting β₂ agonists include, but are not limited to, albuterol, bitolterol, pirbuterol, and terbutaline. Some of the adverse effects associated with the administration of short-acting β₂ agonists include tachycardia, skeletal muscle tremor, hypokalemia, increased lactic acid, headache, and hyperglycemia.

Chromolyn sodium and nedocromil are used as long-term control medications for preventing primarily asthma symptoms arising from exercise or allergic symptoms arising from allergens. These compounds are believed to block early and late reactions to allergens by interfering with chloride channel function. They also stabilize mast cell membranes and inhibit activation and release of mediators from inosineophils and epithelial cells. A four to six week period of administration is generally required to achieve a maximum benefit.

Anticholinergics are generally used for the relief of acute bronchospasm. These compounds are believed to function by competitive inhibition of muscarinic cholinergic receptors. Anticholinergics include, but are not limited to, ipratropium bromide. These compounds reverse only cholinerigically-mediated bronchospasm and do not modify any reaction to antigen. Side effects include drying of the mouth and respiratory secretions, increased wheezing in some individuals, and blurred vision if sprayed in the eyes.

The modified A-class oligonucleotides of the invention may also be useful for treating airway remodeling. Airway remodeling results from smooth muscle cell proliferation and/or submucosal thickening in the airways, and ultimately causes narrowing of the airways leading to restricted airflow. The modified A-class oligonucleotides of the invention may prevent further remodeling and possibly even reduce tissue build-up resulting from the remodeling process.

In one aspect the invention provides a method of treating a subject having an immune system deficiency. The method according to this aspect of the invention includes the step of administering to the subject an effective amount of a composition of the invention to treat the subject. An “immune system deficiency” as used herein refers to a disease or disorder in which the subject's immune system is not functioning in normal capacity or in which it would be useful to boost the subject's immune response, for example to eliminate a tumor or cancer or an infection in the subject. Subjects having an immune deficiency include subjects having an acquired immune deficiency as well as subjects having a congenital immune system deficiency. Subjects having acquired immune deficiency include, without limitation, subjects having a chronic inflammatory condition, subjects having chronic renal insufficiency or renal failure, subjects having infection, subjects having cancer, subjects receiving immunosuppressive drugs, subjects receiving other immunosuppressive treatment, and subjects with malnutrition. In one embodiment the subject has a suppressed CD4+ T-cell population. In one embodiment the subject has an infection with human immunodeficiency virus (HIV) or has acquired immunodeficiency syndrome (AIDS). The method according to this aspect of the invention thus provides a method for boosting an immune response or boosting the ability to mount an immune response in a subject in need of a more vigorous immune response.

The compositions of the invention may also be administered with non-nucleic acid adjuvants. A non-nucleic acid adjuvant is any molecule or compound except for the modified A-class oligonucleotides described herein which can stimulate the humoral and/or cellular immune response. Non-nucleic acid adjuvants include, for instance, adjuvants that create a depo effect, immune stimulating adjuvants, and adjuvants that create a depo effect and stimulate the immune system.

The modified A-class oligonucleotides are also useful as mucosal adjuvants. It has previously been discovered that both systemic and mucosal immunity are induced by mucosal delivery of CpG oligonucleotides. Thus, the oligonucleotides may be administered in combination with other mucosal adjuvants.

Immune responses can also be induced or augmented by the co-administration or co-linear expression of cytokines (Bueler & Mulligan, 1996; Chow et al., 1997; Geissler et al., 1997; Iwasaki et al., 1997; Kim et al., 1997) or co-stimulatory molecules such as B7 (Iwasaki et al., 1997; Tsuji et al., 1997) with the modified A-class oligonucleotides. The term cytokine is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to IP-10, IL-1, IL-2, IL-4, IL-5, IL-6, IL-7, IL-10, IL-12, IL-15, IL-18, granulocyte-macrophage colony stimulating factor (GM-CSF), granulocyte colony stimulating factor (G-CSF), interferon-γ (IFN-γ), IFN-α, tumor necrosis factor (TNF), TGF-β, FLT-3 ligand, and CD40 ligand. In addition to cytokines the CpG oligonucleotides may be used in combination with antibodies against certain cytokines, such as anti-IL-10 and anti-TGF-β, as well as Cox inhibitors, i.e. COX-1 and COX-2 inhibitors.

The modified A-class oligonucleotides of the invention are also useful for improving survival, differentiation, activation and maturation of dendritic cells. The immunostimulatory oligonucleotides have the unique capability to promote cell survival, differentiation, activation and maturation of dendritic cells.

Modified A-class oligonucleotides of the invention also increase natural killer cell lytic activity and antibody-dependent cellular cytotoxicity (ADCC). ADCC can be performed using a modified A-class oligonucleotide in combination with an antibody specific for a cellular target, such as a cancer cell. When the modified A-class oligonucleotide is administered to a subject in conjunction with the antibody, the subject's immune system is induced to kill the tumor cell. The antibodies useful in the ADCC procedure include antibodies which interact with a cell in the body. Many such antibodies specific for cellular targets have been described in the art and many are commercially available. In one embodiment the antibody is an IgG antibody.

In certain aspects the invention provides a method for enhancing epitope spreading. “Epitope spreading” as used herein refers to the diversification of epitope specificity from an initial focused, dominant epitope-specific immune response, directed against a self or foreign protein, to subdominant and/or cryptic epitopes on that protein (intramolecular spreading) or other proteins (intermolecular spreading). Epitope spreading results in multiple epitope-specific immune responses.

The immune response consists of an initial magnification phase, which can either be deleterious, as in autoimmune disease, or beneficial, as in vaccinations, and a later down-regulatory phase to return the immune system to homeostasis and generate memory. Epitope spreading may be an important component of both phases. The enhancement of epitope spreading in the setting of a tumor allows the subject's immune system to determine additional target epitopes, not initially recognized by the immune system in response to an original therapeutic protocol, while reducing the possibility of escape variants in the tumor population and thus affect progression of disease.

The oligonucleotides of the invention may be useful for promoting epitope spreading in therapeutically beneficial indications such as cancer, viral and bacterial infections, and allergy. The method in one embodiment includes the steps of administering a vaccine that includes an antigen and an adjuvant to a subject and subsequently administering to the subject at least two doses of a modified A-class oligonucleotide of the invention in an amount effective to induce multiple epitope-specific immune responses. The method in one embodiment includes the steps of administering a vaccine that includes a tumor antigen and an adjuvant to a subject and subsequently administering to the subject at least two doses of a modified A-class oligonucleotide of the invention in an amount effective to induce multiple epitope-specific immune responses. The method in one embodiment involves applying a therapeutic protocol which results in immune system antigen exposure in a subject, followed by at least two administrations of an immunostimulatory oligonucleotide of the invention, to induce multiple epitope-specific immune responses, i.e., to promote epitope spreading. In various embodiments the therapeutic protocol is surgery, radiation, chemotherapy, other cancer medicaments, a vaccine, or a cancer vaccine.

The therapeutic protocol may be implemented in conjunction with an immunostimulant, in addition to the subsequent immunostimulant therapy. For instance, when the therapeutic protocol is a vaccine, it may be administered in conjunction with an adjuvant. The combination of the vaccine and the adjuvant may be a mixture or separate administrations, i.e., injections (i.e., same drainage field). Administration is not necessarily simultaneous. If non-simultaneous injection is used, the timing may involve pre-injection of the adjuvant followed by the vaccine formulation.

After the therapeutic protocol is implemented, immunostimulant monotherapy begins. The optimized frequency, duration, and site of administration will depend on the target and other factors, but may for example be a monthly to bi-monthly administration for a period of six months to two years. Alternatively the administration may be on a daily, weekly, or biweekly basis, or the administration may be multiple times during a day, week or month. In some instances, the duration of administration may depend on the length of therapy, e.g., it may end after one week, one month, after one year, or after multiple years. In other instances the monotherapy may be continuous as with an intravenous drip. The immunostimulant may be administered to a drainage field common to the target.

For use in therapy, different doses may be necessary for treatment of a subject, depending on activity of the compound, manner of administration, purpose of the immunization (i.e., prophylactic or therapeutic), nature and severity of the disorder, age and body weight of the subject. The administration of a given dose can be carried out both by single administration in the form of an individual dose unit or else several smaller dose units. Multiple administration of doses at specific intervals of weeks or months apart is usual for boosting antigen-specific immune responses.

Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic agent being administered, the size of the subject, or the severity of the disease or condition. One of ordinary skill in the art can empirically determine the effective amount of a particular nucleic acid and/or other therapeutic agent without necessitating undue experimentation.

Subject doses of the compounds described herein typically range from about 0.1 μg to 10,000 mg, more typically from about 1 pg/day to 8000 mg, and most typically from about 10 pg to 100 pg. Stated in terms of subject body weight, typical dosages range from about 0.1 μg to 20 mg/kg/day, more typically from about 1 to 10 mg/kg/day, and most typically from about 1 to 5 mg/kg/day.

The pharmaceutical compositions containing nucleic acids and/or other compounds can be administered by any suitable route for administering medications. A variety of administration routes are available. The particular mode selected will depend, of course, upon the particular agent or agents selected, the particular condition being treated, and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration are discussed herein. For use in therapy, an effective amount of the nucleic acid and/or other therapeutic agent can be administered to a subject by any mode that delivers the agent to the desired surface, e.g., mucosal, systemic.

Administering the pharmaceutical composition of the present invention may be accomplished by any means known to the skilled artisan. Routes of administration include but are not limited to oral, parenteral, intravenous, intramuscular, intraperitoneal, intranasal, sublingual, intratracheal, inhalation, subcutaneous, ocular, vaginal, and rectal. For the treatment or prevention of asthma or allergy, such compounds are preferably inhaled, ingested or administered by systemic routes. Systemic routes include oral and parenteral. Inhaled medications are preferred in some embodiments because of the direct delivery to the lung, the site of inflammation, primarily in asthmatic patients. Several types of devices are regularly used for administration by inhalation. These types of devices include metered dose inhalers (MDI), breath-actuated MDI, dry powder inhaler (DPI), spacer/holding chambers in combination with MDI, and nebulizers.

The therapeutic agents of the invention may be delivered to a particular tissue, cell type, or to the immune system, or both, with the aid of a vector. In its broadest sense, a “vector” is any vehicle capable of facilitating the transfer of the compositions to the target cells. The vector generally transports the immunostimulatory nucleic acid, antibody, antigen, and/or disorder-specific medicament to the target cells with reduced degradation relative to the extent of degradation that would result in the absence of the vector.

In general, the vectors useful in the invention are divided into two classes: biological vectors and chemical/physical vectors. Biological vectors and chemical/physical vectors are useful in the delivery and/or uptake of therapeutic agents of the invention.

Most biological vectors are used for delivery of nucleic acids and this would be most appropriate in the delivery of therapeutic agents that are or that include immunostimulatory nucleic acids.

In addition to the biological vectors discussed herein, chemical/physical vectors may be used to deliver therapeutic agents including immunostimulatory nucleic acids, antibodies, antigens, and disorder-specific medicaments. As used herein, a “chemical/physical vector” refers to a natural or synthetic molecule, other than those derived from bacteriological or viral sources, capable of delivering the nucleic acid and/or other medicament.

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vesicles (LUVs), which range in size from 0.2-4.0 μm can encapsulate large macromolecules. RNA, DNA and intact virions can be encapsulated within the aqueous interior and be delivered to cells in a biologically active form. Fraley et al. (1981) Trends Biochem Sci 6:77.

Liposomes may be targeted to a particular tissue by coupling the liposome to a specific ligand such as a monoclonal antibody, sugar, glycolipid, or protein. Ligands which may be useful for targeting a liposome to an immune cell include, but are not limited to: intact or fragments of molecules which interact with immune cell specific receptors and molecules, such as antibodies, which interact with the cell surface markers of immune cells. Such ligands may easily be identified by binding assays well known to those of skill in the art. In still other embodiments, the liposome may be targeted to the cancer by coupling it to a one of the immunotherapeutic antibodies discussed earlier. Additionally, the vector may be coupled to a nuclear targeting peptide, which will direct the vector to the nucleus of the host cell.

Lipid formulations for transfection are commercially available from QIAGEN, for example, as EFFECTENE™ (a non-liposomal lipid with a special DNA condensing enhancer) and SUPERFECT™ (a novel acting dendrimeric technology).

Liposomes are commercially available from Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes also have been reviewed by Gregoriadis G (1985) Trends Biotechnol 3:235-241.

Certain cationic lipids, including in particular N-[1-(2,3 dioleoyloxy)-propyl]-N,N,N-trimethylammonium methyl-sulfate (DOTAP), appear to be especially advantageous when combined with the modified oligonucleotide analogs of the invention.

In one embodiment, the vehicle is a biocompatible microparticle or implant that is suitable for implantation or administration to the mammalian recipient. Exemplary bioerodible implants that are useful in accordance with this method are described in PCT International application no. PCT/US/03307 (Publication No. WO95/24929, entitled “Polymeric Gene Delivery System”. PCT/US/0307 describes a biocompatible, preferably biodegradable polymeric matrix for containing an exogenous gene under the control of an appropriate promoter. The polymeric matrix can be used to achieve sustained release of the therapeutic agent in the subject.

The polymeric matrix preferably is in the form of a microparticle such as a microsphere (wherein the nucleic acid and/or the other therapeutic agent is dispersed throughout a solid polymeric matrix) or a microcapsule (wherein the nucleic acid and/or the other therapeutic agent is stored in the core of a polymeric shell). Other forms of the polymeric matrix for containing the therapeutic agent include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and the nucleic acid and/or the other therapeutic agent are encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time. In some preferred embodiments, the nucleic acid are administered to the subject via an implant while the other therapeutic agent is administered acutely. Biocompatible microspheres that are suitable for delivery, such as oral or mucosal delivery, are disclosed in Chickering et al. (1996) Biotech Bioeng 52:96-101 and Mathiowitz E et al. (1997) Nature 386:410-414 and PCT Pat. Application WO97/03702.

Both non-biodegradable and biodegradable polymeric matrices can be used to deliver the nucleic acid and/or the other therapeutic agent to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable, particularly for the nucleic acid agents. The polymer optionally is in the form of a hydrogel that can absorb up to about 90% of its weight in water and further, optionally is cross-linked with multi-valent ions or other polymers.

Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

If the therapeutic agent is a nucleic acid, the use of compaction agents may also be desirable. Compaction agents also can be used alone, or in combination with, a biological or chemical/physical vector. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver a nucleic acid in a form that is more efficiently taken up by the cell or, more preferably, in combination with one or more of the above-described vectors.

Other exemplary compositions that can be used to facilitate uptake of a nucleic acid include calcium phosphate and other chemical mediators of intracellular transport, microinjection compositions, electroporation and homologous recombination compositions (e.g., for integrating a nucleic acid into a preselected location within the target cell chromosome).

The compounds may be administered alone (e.g., in saline or buffer) or using any delivery vehicle known in the art. For instance the following delivery vehicles have been described: cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); liposomes (Childers et al., 1999, Michalek et al., 1989, 1992, de. Haan 1995a, 1995b); live bacterial vectors (e.g., Salmonella, Escherichia coli, Bacillus Calmette-Guérin, Shigella, Lactobacillus) (Hone et al., 1996, Pouwels et al., 1998, Chatfield et al., 1993, Stover et al., 1991, Nugent et al., 1998); live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); microspheres (Gupta et al., 1998, Jones et al., 1996, Maloy et al., 1994, Moore et al., 1995, O'Hagan et al., 1994, Eldridge et al., 1989); nucleic acid vaccines (Fynan et al., 1993, Kuklin et al., 1997, Sasaki et al., 1998, Okada et al., 1997, Ishii et al., 1997); polymers (e.g. carboxymethylcellulose, chitosan) (Hamajima et al., 1998, Jabbal-Gill et al., 1998); polymer rings (Wyatt et al., 1998); proteosomes (Vancott et al., 1998, Lowell et al., 1988, 1996, 1997); sodium fluoride (Hashi et al., 1998); transgenic plants (Tacket et al., 1998, Mason et al., 1998, Haq et al., 1995); virosomes (Gluck et al., 1992, Mengiardi et al., 1995, Cryz et al., 1998); and, virus-like particles (Jiang et al., 1999, Leibl et al., 1998).

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency.

For oral administration, the compounds (i.e., nucleic acids, antigens, antibodies, and other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long-acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R (1990) Science 249:1527-1533, which is incorporated herein by reference.

The nucleic acids and optionally other therapeutics and/or antigens may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di-, and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

### Derivitization of A Class ODN SEQ ID NO:2 Results in ODN with Increased Ability to Induce IFN-α In Vitro

The G-rich mixed backbone oligonucleotide SEQ ID NO:2 has been demonstrated to be very effective in inducing IFN-α secretion, and thus could be used to treat those human diseases in which a strong IFN-α response would be beneficial, such as cancer and infectious diseases. However, development of this oligonucleotide has been hampered by certain issues connected with the biophysical properties of this class of compound, such as tendency to aggregation, poor solubility, difficulties in quality control and solid phase extraction (SPE) used in PK studies. SEQ ID NO:2 is characterized by its very efficient induction of IFN-α secretion, but low B cell stimulation. As such it is classified as an A-class oligonucleotide. SEQ ID NO:2 consists of a palindromic phosphodiester CpG sequence (ACG ACG TCG T) clamped by phosphorothioate (G)n stretches.

| | |
|---|---|
| SEQ ID NO:2 | 5′-G*G*G-G-A-C-G-A-C-G-T-C-G-T-G-G* |
| | G*G*G*G*G |

(* is phosphorothioate, - is phosphodiester)

In an attempt to discover new oligonucleotides having the potency of SEQ ID NO:2 but with more favorable biophysical properties compared to this G-rich ODN, a series of oligonucleotides with reduced G content and a reduced number of phosphorothioate linkages was designed and tested.

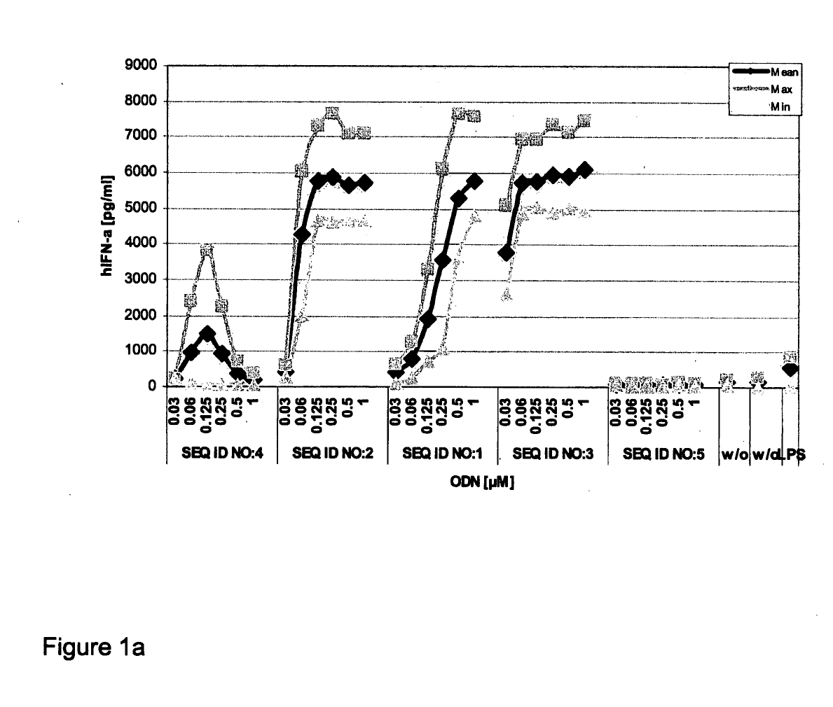

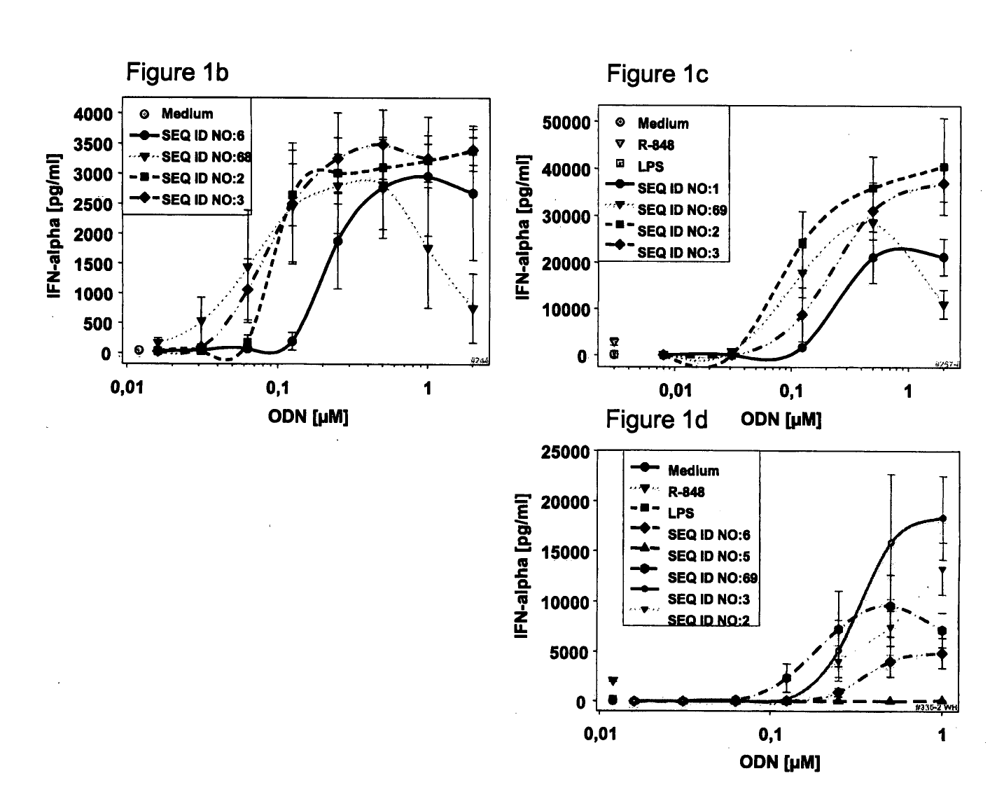

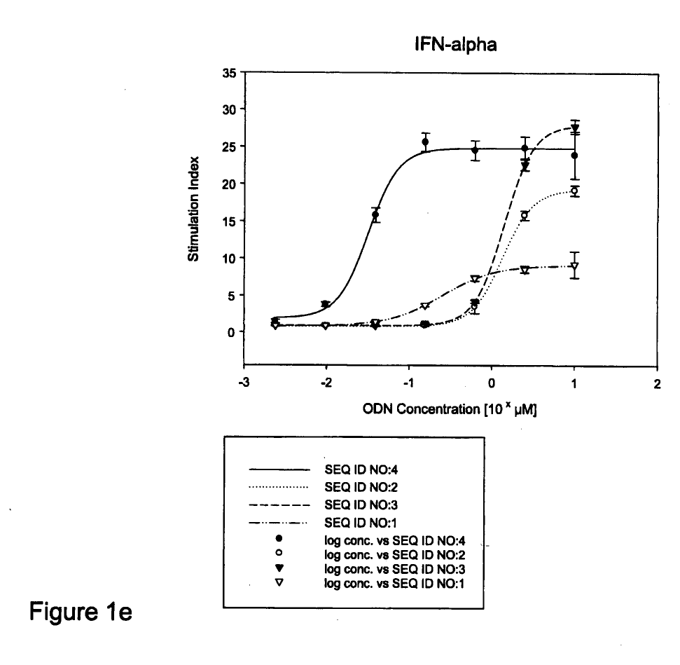

ODN with a 5′-TCG motif are usually recognized by TLR9. Therefore, the 10 nucleotide ACG ACG TCG T palindrome of SEQ ID NO:2 was converted into the 8 nucleotide palindrome TCG ACG TCG T (see SEQ ID NO:3, table 2). To test this shortened ODN, human peripheral blood mononuclear cells (PBMCs) were isolated from healthy donors, plated, and stimulated in vitro with various test and control immunostimulatory agents for 48 hours. After 48 hours, the supernatants were collected and then analyzed by ELISA assay. Surprisingly, the shortened palindrome sequence present in ODN SEQ ID NO:3 gave a much higher IFN-α induction as compared to a sequence containing the entire 10 nucleotides palindrome of SEQ ID NO:2. The induction of IFN-α secretion by the SEQ ID NO:3 (15 nucleotides in length) was equal to (FIGS. 1a-1c) or better than that of SEQ ID NO:2 (21 nucleotides in length) (FIG. 1d). SEQ ID NO:2 and 3 were also better at inducing IFN-α than B-class (SEQ ID NO:4) and double palindromic C or P class (SEQ ID NO:1, 68, 69).

FIG. 1e shows the ability of SEQ ID NO:3 to stimulate TLR9. Stably transfected HEK293 cells expressing the human TLR9 or murine TLR9 were described before. Briefly, HEK293 cells were transfected by electroporation with vectors expressing the respective TLR and a 6×NF-κB-luciferase reporter plasmid. Stable transfectants (3×10⁴ cells/well) were incubated with ODN for 16 h at 37° C. in a humidified incubator. Each data point was done in triplicate. Cells were lysed and assayed for luciferase gene activity (using the BriteLite kit from Perkin-Elmer, Zaventem, Belgium). Stimulation indices were calculated in reference to reporter gene activity of medium without addition of ODN. EC₅₀ values were calculated using the Sigma Plot program (SSPS Inc.) using sigmoidal regression curves (4 parameters). Again, SEQ ID NO:3 stimulated TLR9 activity to a greater degree than the ODN with the longer palindrome, SEQ ID NO:2.

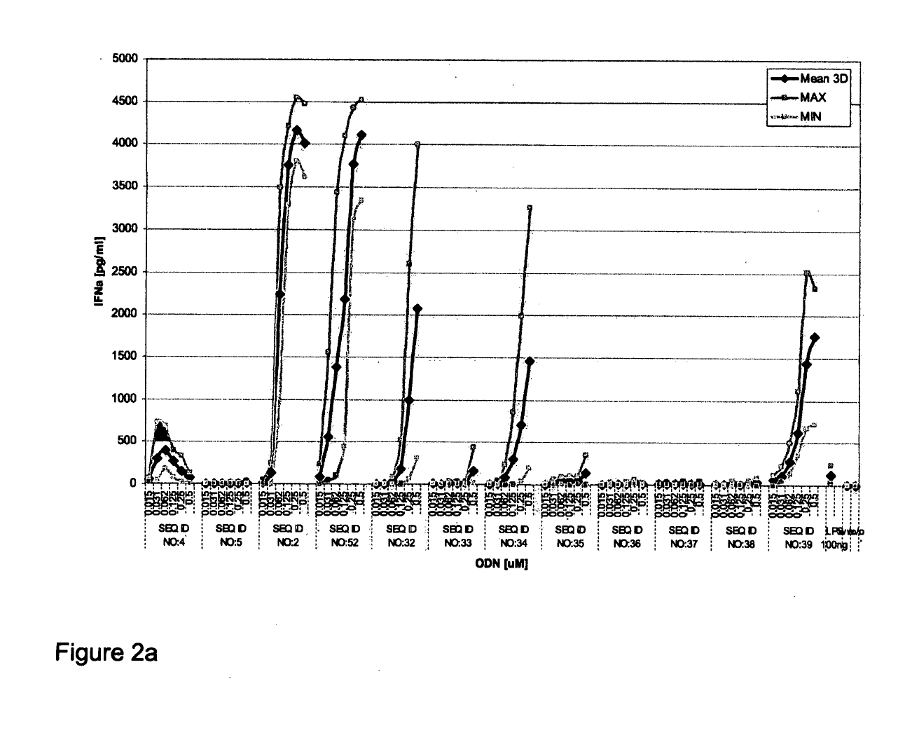

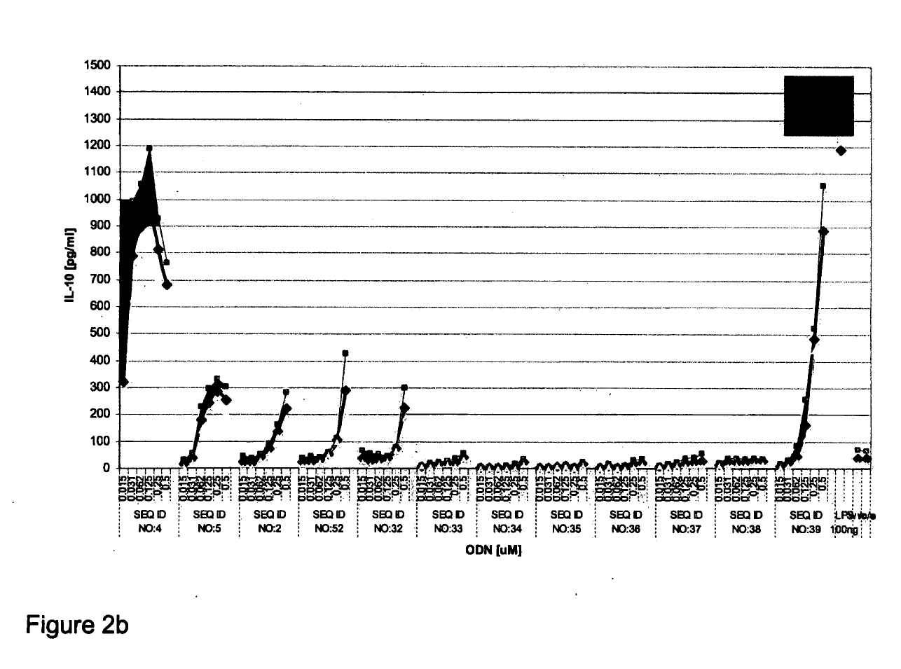

A number of derivatives of SEQ ID NO:2 were made and tested for their ability to induce IFN-α and IL-10. In addition to SEQ ID NO:3, also tested were one semi-soft ODN (SEQ ID NO:32) and its fully phosphorothioate counterpart (SEQ ID NO:33), an ODN containing the full palindrome of SEQ ID NO:2 (SEQ ID NO:34) and two ODN containing a defect in the palindrome sequence (SEQ ID NO: 35-36), and three ODN with the G₅ sequence interrupted (SEQ ID NO:38) or reduced to G₄ (SEQ ID NO:37 and 39) (see Table 2). As shown in FIG. 2a, the semi-soft oligonucleotide with the sequence similar to SEQ ID NO:3, SEQ ID NO:32, resulted in the greatest IFN-α stimulation. Even with the full palindromic sequence of SEQ ID NO:2, SEQ ID NO:34 was less active than SEQ ID NO:2. A G₄ sequence alone was not sufficient for activity, as SEQ ID NO:37 was not active but SEQ ID NO:39 was. As shown in FIG. 2b, none of the ODN were capable of inducing significant IL-10 except for SEQ ID NO:32 and, surprisingly, SEQ ID NO:39 which showed a very strong IL-10 induction.

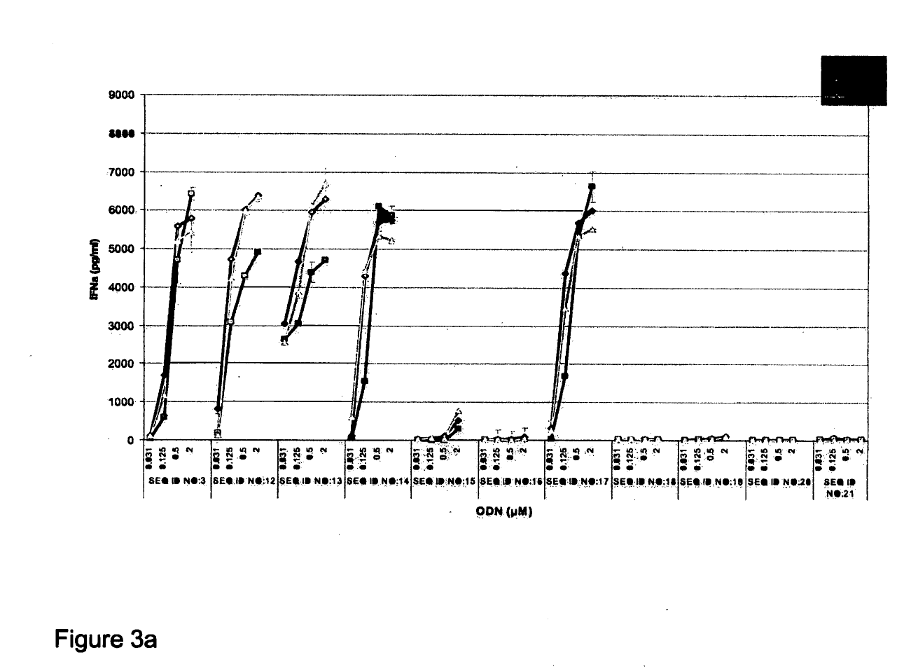

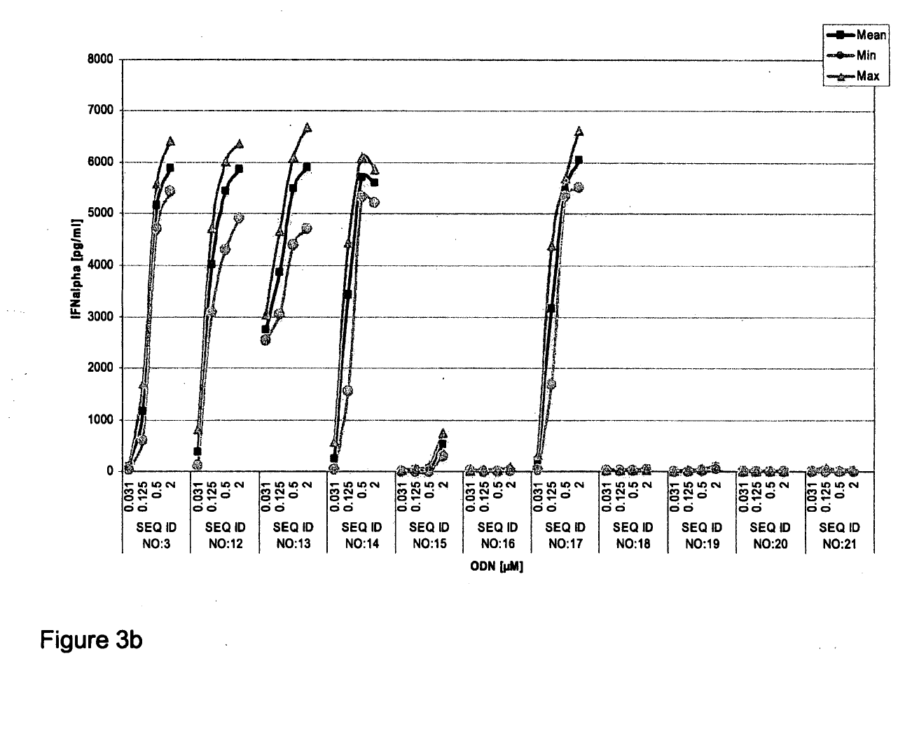

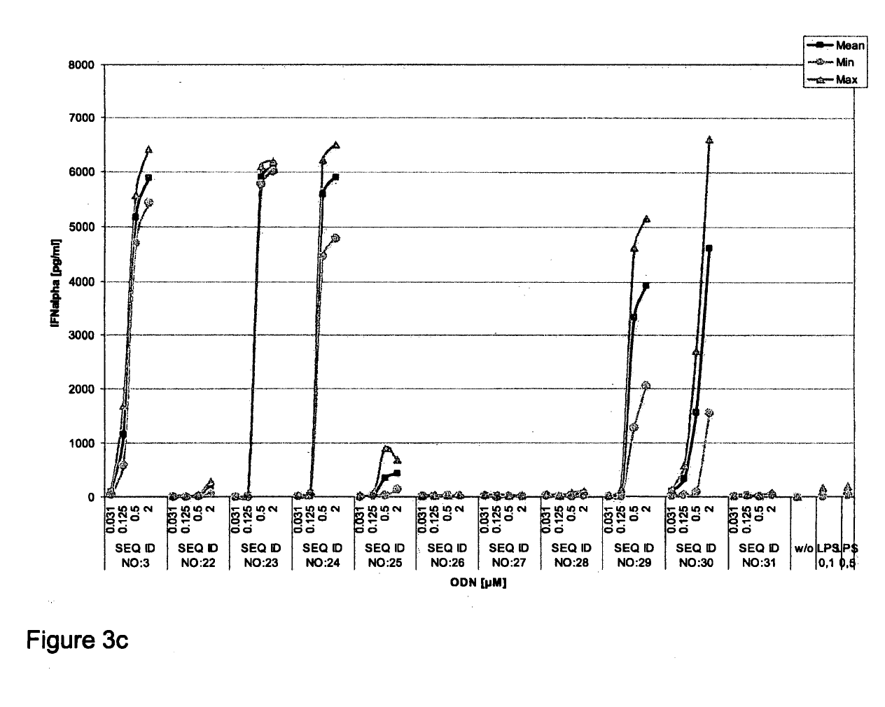

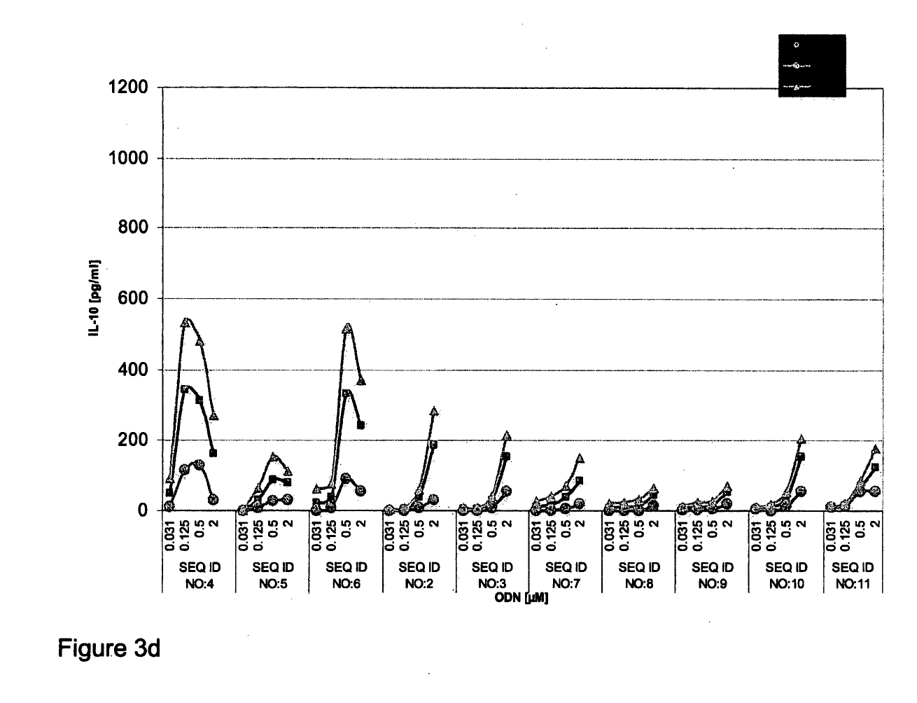

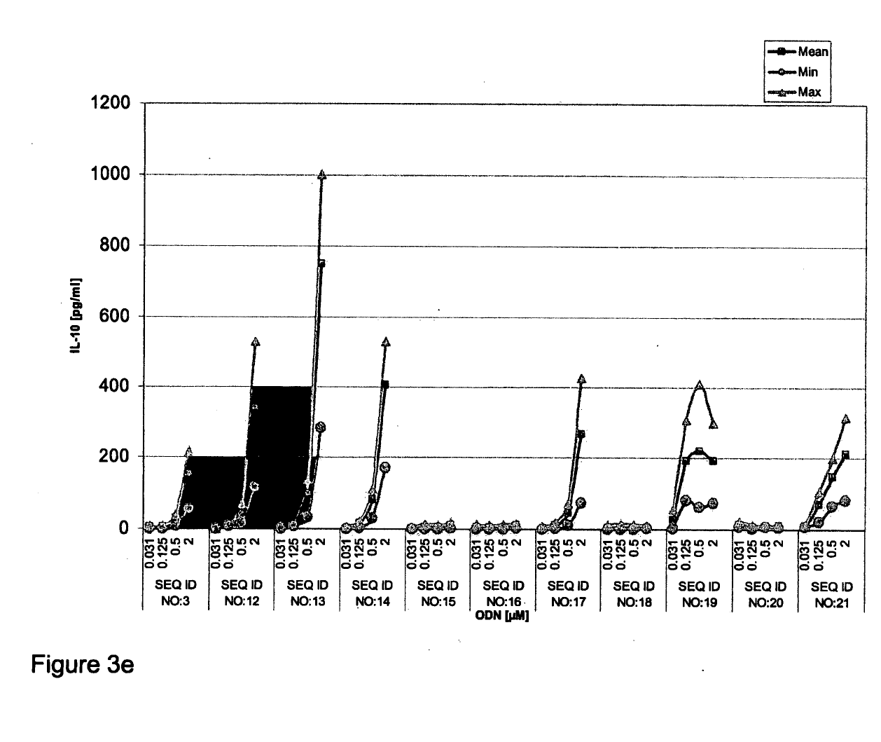

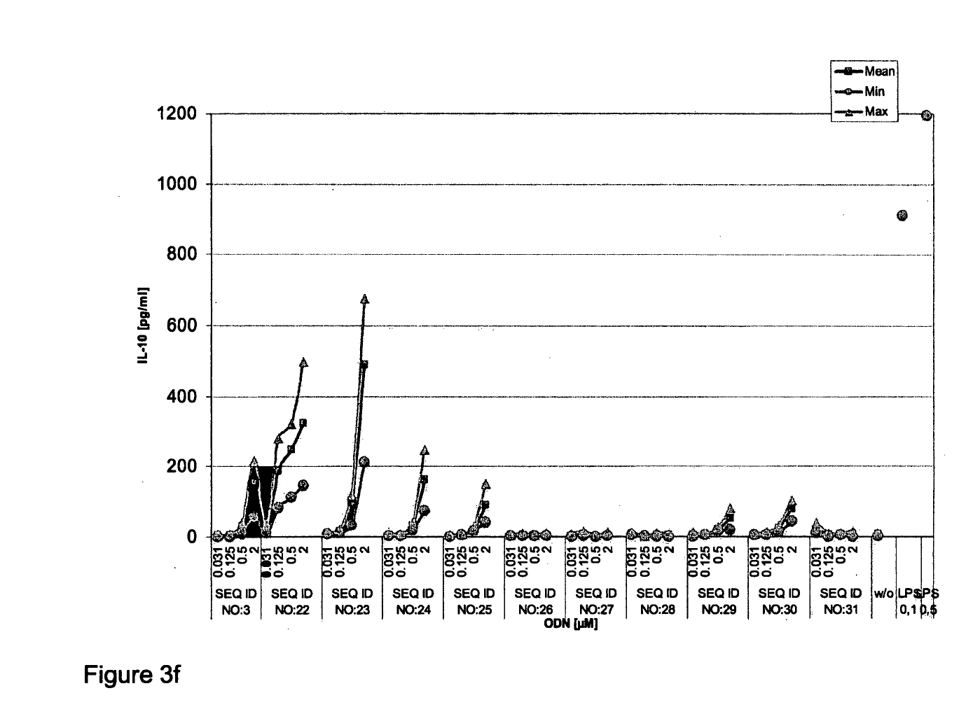

A number of oligonucleotides were designed based on the data shown in FIG. 2 (SEQ ID NO:7-31). Of these, SEQ ID NO:13 showed the strongest ability to induce both IFN-α (FIGS. 3a-3c) and IP-10 (FIGS. 3d-3f).

TABLE 2

| SEQ ID | | IFN-α |
|---|---|---|
| Number | SEQ ID NO: 2 Derivative | induction |
| 2 | G*G*G_G_A_C_G_A_C_G_T_C_G_T_G_ | ++++ |
| | G*G*G*G*G*G | |
| 3 | T*C_G_A_C_G_T_C_G_T_G_G*G*G*G | |
| 7 | T*C_G_T_C_G_A_C_G_T_G_G*G*G* | +++++ |
| 8 | T*C_G_C_C_G_G_C_G_T_G_G*G*G*G | +++ |
| 9 | T*C_G_G_C_G_C_C_G_T_G_G*G*G*G | +++ |
| 10 | T*C_G_A_C_G_T_C_G_A_C_G_T_C_G_ | ++++ |
| | T_G_G*G*G*G | |
| 11 | T*C_G_A_C_G_T_C_G_T_T_G_G*G*G*G | ++++ |
| 12 | G*T*C_G_A_C_G_T_C_G_T_G_G*G*G*G | ++++ |
| 13 | G*T*C_G_A_C_G_T_C_G_T_T_G_G*G*G*G | +++++ |
| 14 | T*C_G_T_C_G_A_C_G_T_T_G_G*G*G*G | ++++ |
| 15 | T*C_G_A_C_G_T_C_G_T_G_G*G*T*G | + |
| 16 | T*C_G_A_C_G_T_C_G_T_G_T*T*T*T | − |
| 17 | T*C_G_A_C_G_T_C_G_T_G_G_G*G*G | ++++ |
| 18 | T*C_G_A_C_G_T_C_G*T | + |
| 19 | A*C*G*A*C*G*T*T*T*T*G*T*C*G*T* | 0 |
| | T*T*T*G*T*C*G*T*T | |
| 20 | A*C_G_A_C_G_T_C_G*T | 0 |
| 21 | A*C_G_A_C_G_T_C_G*T*T*T*T*T*T* | 0 |
| | T*T*T*T*T | |
| 22 | A*C*G*A*C*G*T*C*G*T*T*T*T*T*T* | 0 |
| | T*T*T*T*T | |
| 23 | G*G*G_G_T*C_G_A_C_G_T_C_G_T_G_ | ++++ |
| | G*G*G*G*G*G | |
| 24 | G*G*G_G_T*C_G_A_C_G_T_C_G_T_G_ | ++++ |
| | G*G*G*G | |
| 25 | G*G*G_G_T_C_G_T_C_G_T_C_G_T_G_ | + |
| | G*G*G*G*G*G | |
| 26 | G*G_G_T_G_G_G_T_G_G_G_T_G_G_G*T | 0 |
| 27 | G*G_C_G_T_G_G_C_G_T_G_G_C_G_T_G_ | 0 |
| | G_C_G*T | |
| 28 | G*G_C_G_T_C_G_G_C_G_T_C_G_G_C_G_ | 0 |
| | T_C_G_G_C_G*T | |
| 29 | I*C_G_A_C_G_T_C_G_T_G_G*G*G*G | ++ |
| 30 | T*C_G_A_C_G_T_C_G_T_G_G_G_G_G*T | ++++ |
| 31 | T_C_G_A_C_G_T_C_G_T_D_D_D_D_T_ | 0 |
| | C_G_A_C_G_T_C_G_T_D_D_D | |
| 32 | T*C_G*A*C_G*T*C_G*T_G_G*G*G*G | +++ |
| 33 | T*C*G*A*C*G*T*C*G*T*G*G*G*G*G | 0 |
| 34 | A*C_G_A_C_G_T_C_G_T_G_G*G*G*G | ++ |
| 35 | T*C_G_A_C_G_A_C_G_T_G_G*G*G*G | 0 |
| 36 | A*C_G_T_C_G_T_C_G_T_G_G*G*G*G | 0 |
| 37 | T*C_G_A_C_G_T_C_G_T_C_G*G*G*G | 0 |
| 38 | T*C_G_A_C_G_T_C_G_T_G_G*T*G*G | 0 |
| 39 | T*C_G_A_C_G_T_C_G_T_G_G*G*G | ++ |
| 40 | T*C_G_A_C_G_T_C_G_T_hex | + |
| 41 | T*C_G_A_C_G_T_C_G_T_teg | − |
| 42 | T*C_G_A_C_G_T_C_G*T_Chol | ++ |
| 43 | T_C_G_A_C_G_T_C_G_T_Chol | +++ |
| 44 | Chol_T_C_G_A_C_G_T_C_G_T_Chol | + |

Key
chol cholesterol
teg triethylene glycol
hex hexadecyl glyceryl ether
_ phosphodiester internucleotide bond
* phosphorothioate internucleotide bond

### Example 2

### Lipophilic Derivitization of New A-class ODN

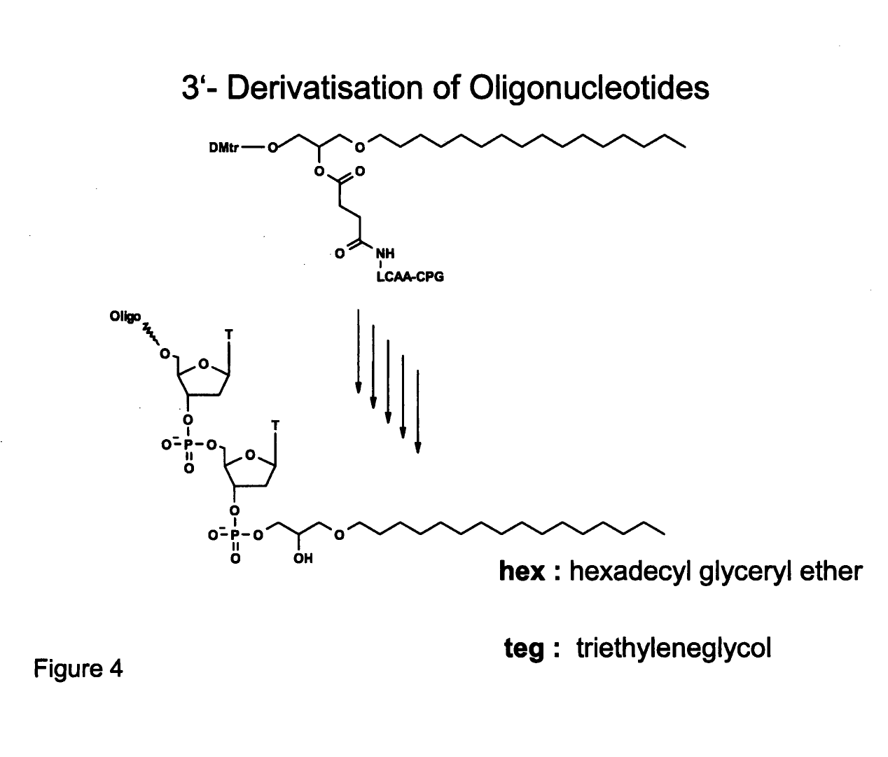

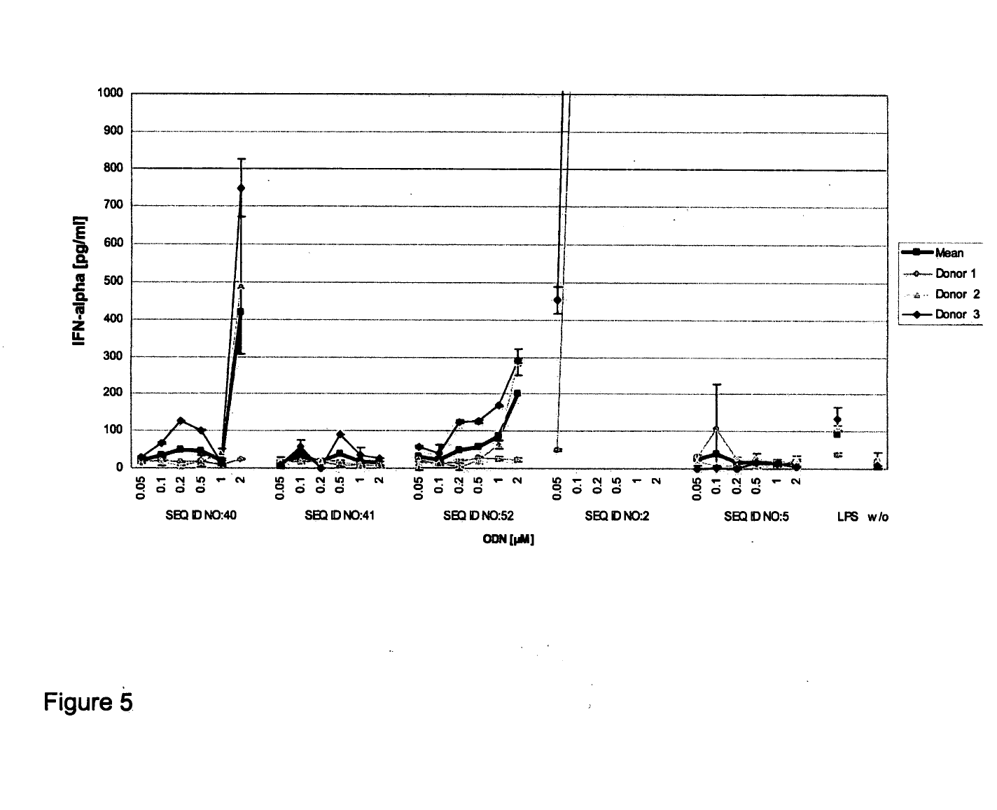

Lipophilic derivatives of SEQ ID NO:3 were derived and tested for their ability to induce IFN-α. A schematic of the process for adding hexadecyl glyceryl ether or triethylene glycol to the 3′ end of ODN is shown in FIG. 4. Two derivatives of SEQ ID NO:3 were synthesized with lipophilic tags in place of the 3′ poly G motif: SEQ ID NO:40, with a hexadecyl glyceryl ether moiety, and SEQ ID NO:41, with a triethylene glycol moiety (see table 2). These ODN were then tested for the ability to induce IFN-α in vitro. As shown in FIG. 5, the ODN with the hexadecyl glyceryl ether tag showed better activity than the ODN with the triethylene glycol tag, although neither one induced as much IFN-α as SEQ ID NO:2. The low activity of the teg-modified ODN (SEQ ID NO:41) is likely due to its low cellular uptake as compared to G-rich (SEQ ID NO:39) ODN or lipophilic-modified ODNs (SEQ ID NO:40 and SEQ ID NO:42). The teg-modified ODN was chosen as a control to show that stabilization of the ODN to 3′-exonucleases by 3′-modification (teg, hex or chol) alone is not sufficient to obtain good biological activity.

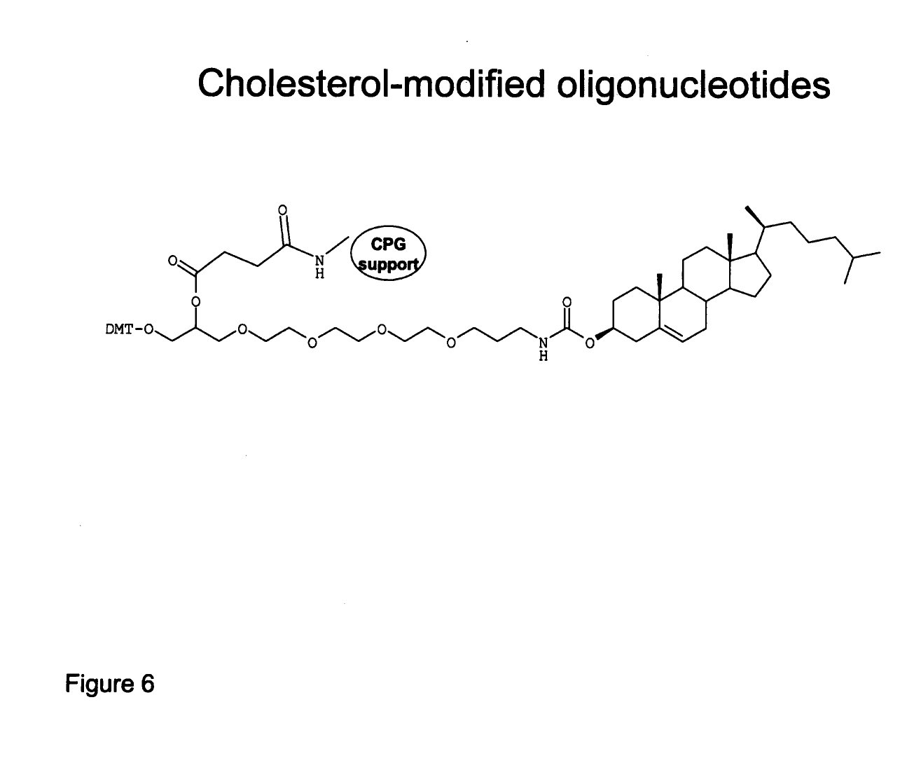

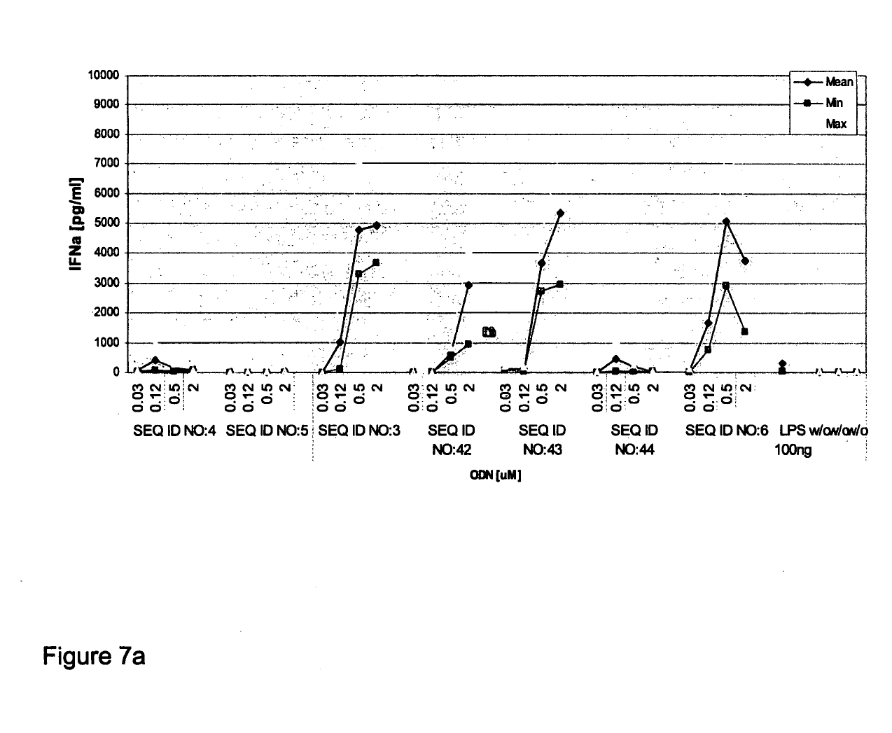

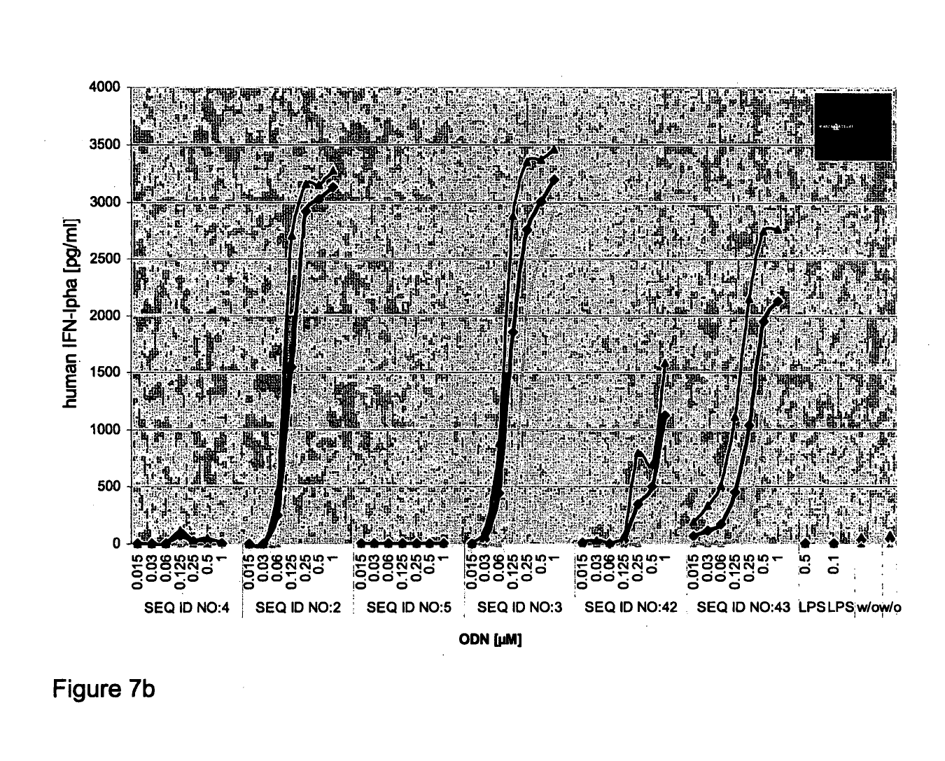

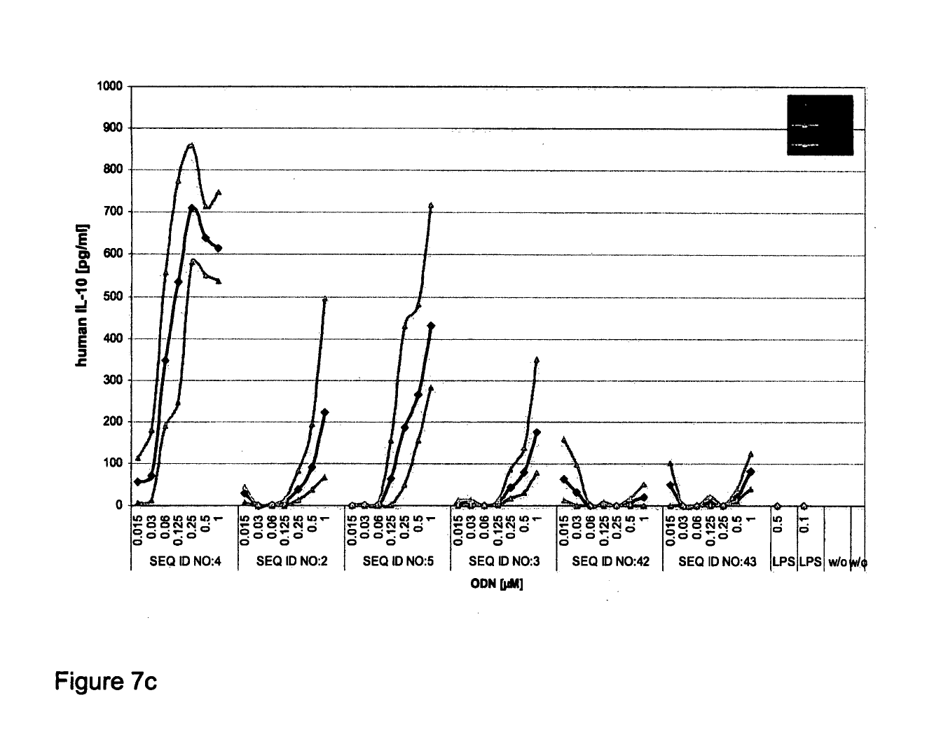

A schematic of the process for adding a cholesterol tag to an ODN is shown in FIG. 6. Three derivatives of SEQ ID NO:3 were synthesized with cholesterol tags. SEQ ID NO:42 has a cholesterol tag in place of the 3′ poly G motif and the terminal bonds of the ODN are phosphorothioate bonds. SEQ ID NO:43 has a phosphodiester backbone and a 3′ cholesterol tag. SEQ ID NO:44 has a phosphodiester backbone and both a 5′ and a 3′ cholesterol tag. Human peripheral blood mononuclear cells (PBMCs) were isolated from healthy donors, plated, and stimulated in vitro with various test and control immunostimulatory agents for 48 hours. After 48 hours, the supernatants were collected and then analyzed by ELISA assay (FIG. 7a). SEQ ID NO:43 induced levels of IFN-α comparable to that of SEQ ID NO:3 or SEQ ID NO:6, a C-class CpG ODN. SEQ ID NO:42 induced IFN-α less well, and SEQ ID NO:44 did not induce a significant amount of IFN-α. This process was repeated for IFN-α (FIG. 7b) and IL-10 (FIG. 7c). Neither SEQ ID NO:42 or 43 induced a significant amount of IL-10.

### Example 3

### In Vivo Cytokine Induction by Modified A-class ODN SEQ ID NO:3 is Dependent Upon Route of Administration

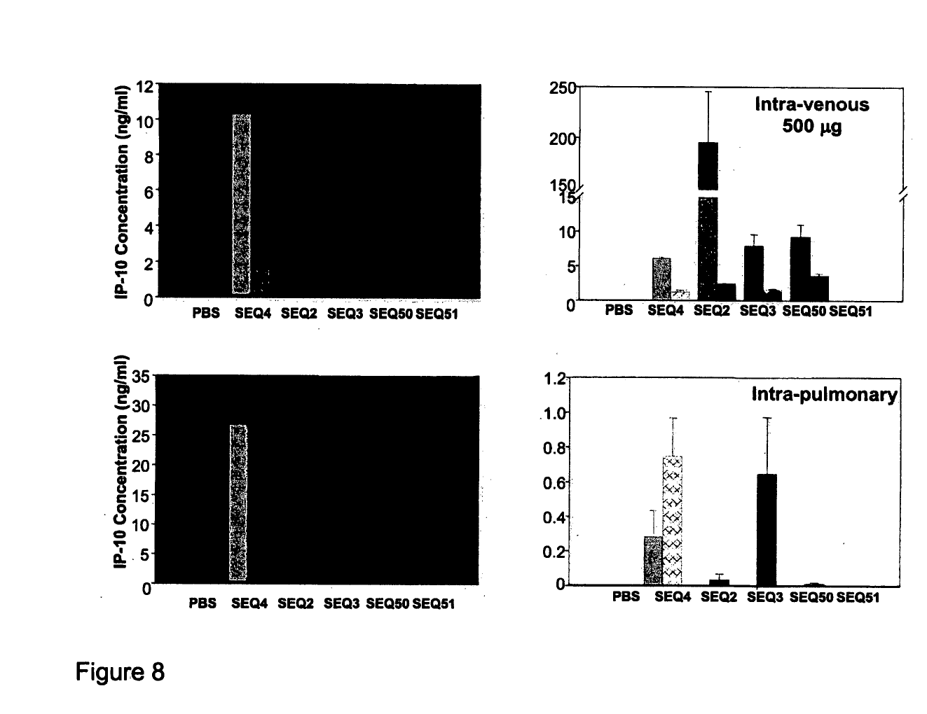

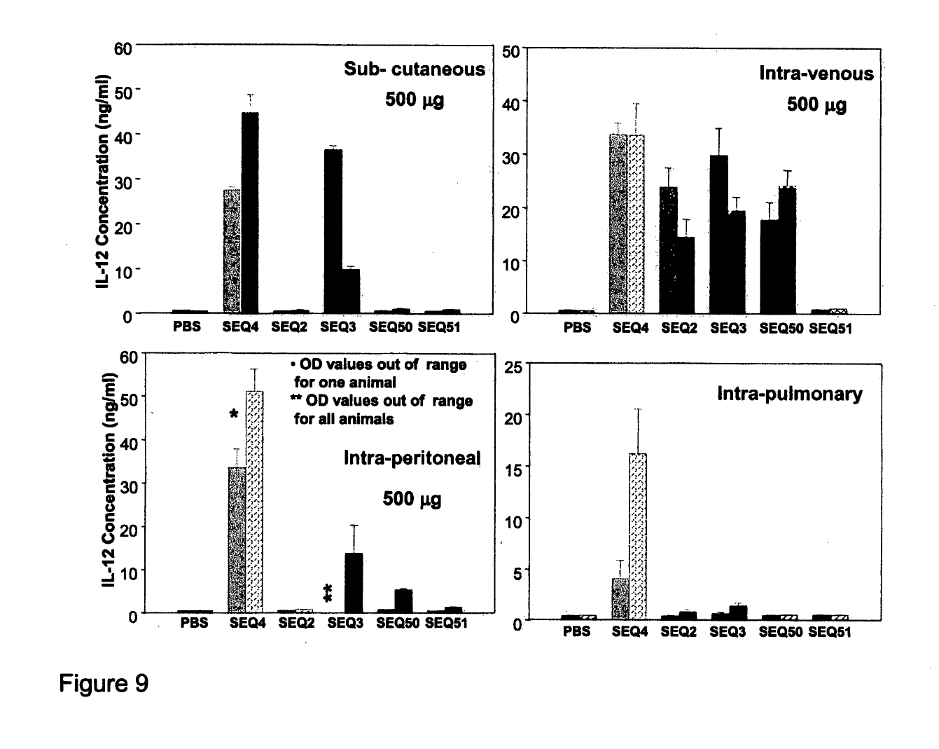

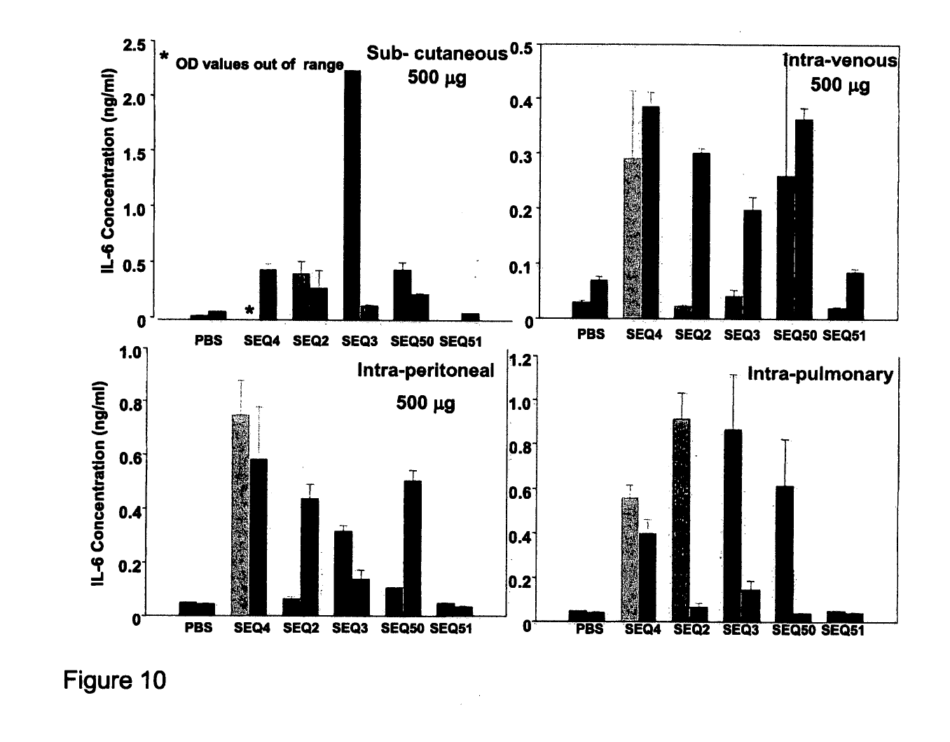

To test the ability of SEQ ID NO:3 to induce an immune response in vivo, Balb/c mice were injected with SEQ ID NO:2-4 as well as SEQ ID NO:50, another A-class ODN, and 51, a negative control ODN. ODN were administered subcutaneously (SC), intravenously (IV), or intra-peritoneally (IP) with 500 pg of the indicated ODN or intra-pulmonary (IPul) with 250 pg of the indicated ODN. FIGS. 8-10 show the resulting cytokine/chemokine stimulation of IP-10, IL-12, and IL-6, respectively. Animals were bled at 3 hours (solid bars) or 8 hours (hatched bars). SEQ ID NO:3 was most effective compared to SEQ ID NO:2 and SEQ ID NO:50 when administered by SC, IP, and IPul routes, except in the case of the IL-6 induction by IP and IPul routes where all three A-class ODN were equally potent. SEQ ID NO:2 was superior to the rest of the A-class ODN tested, as well as the B-class ODN SEQ ID NO:4, in promoting IP-10 induction by IV route.

### Example 4

### Intermolecular Interaction of ODN SEQ ID NO:3

It is known that (G)n stretches in oligonucleotides, where n≧4, lead to intermolecular tetrad formation resulting in non homogeneous high molecular aggregates. The uptake of oligonucleotides with (G)n stretches is about 20 to 40-times higher than of non-aggregated oligonucleotides and the intracellular localization appears also to be different. It is not understood how these observations correlate with biological activity.

When analyzed by capillary gel electrophoresis (CGE) and MALDI-TOF mass spectrometry, ODN SEQ ID NO:3 shows partial dimer formation. UV-thermal denaturation reveals two transitions, suggesting two different structural species in solution. The first species melts with a Tm of 82° C. and the second species melts with a Tm of 41° C. The melting of the first species (82° C.) is observed only when the ODN solution is heated but not on cooling of the previously heated ODN solution. When analyzed by size exclusion chromatography (SEC), SEQ ID NO:2 shows aggregation to high molecular structures resulting in a number of different peaks in SEC. Surprisingly, SEQ ID NO:3 shows only peaks in the low molecular range (likely monomer or dimer) although it contains the GGGGG motif which in principal can still lead to intramolecular tetrad formation. Taken together, ODN SEQ ID NO:3 appears to form an intramolecular tetrad which is stabilized by the 5′-T nucleotide, but not (or significantly less) by the 5′-A nucleotide as present in SEQ ID NO:2. The intramolecular structure consists of two molecules of SEQ ID NO:3 which is stabilized by non-Watson-Crick base-pairing. Alternative sequences may possibly be designed which will fold into similar intramolecular tetrad structures resulting in high IFN-α induction. Likewise, replacement of G or T by alternative nucleosides, which also support tetrad formation (e.g. inosine), may also lead to active ODNs.

A list of modified A-class and other ODN is provided in Table 3.

TABLE 3
Modified A-class and other ODN Sequences

| SEQ ID | |
|---|---|
| Number | Sequence |
| 1 | T*C G*T*C_G*T*T*T*T*G*C_G*C*G*G*C*C*G*C*C*G |
| 2 | G*G*G_G_A_C_G_A_C_G_T_C_G_T_G_G*G*G*G*G*G |
| 3 | T*C_G_A_C_G_T_C_G_T_G_G*G*G*G |
| 4 | T*C_G*T*C_G*T*T*T*T_G*T*C_G*T*T*T*T_G*T* |
| | C_G*T*T |
| 5 | T*C*C*A*G*G*A*C*T*T*C*T*C*T*C*A*G*G*T*T |
| 6 | T*C*G*T*C*G*T*T*T*T*C*G*G*C*G*C*G*C*G*C*C*G |
| 7 | T*C_G_T_C_G_A_C_G_T_G_G*G*G* |
| 8 | T*C_G_C_C_G_G_C_G_T_G_G*G*G*G |
| 9 | T*C_G_G_C_G_C_C_G_T_G_G*G*G*G |
| 10 | T*C_G_A_C_G_T_C_G_A_C_G_T_C_G_T_G_G*G*G*G |
| 11 | T*C_G_A_C_G_T_C_G_T_T_G_G*G*G*G |
| 12 | G*T*C_G_A_C_G_T_C_G_T_G_G_G*G*G |
| 13 | G*T*C_G_A_C_G_T_C_G_T_T_G_G*G*G*G |
| 14 | T*C_G_T_C_G_A_C_G_T_T_G_G*G*G*G |
| 15 | T*C_G_A_C_G_T_C_G_T_G_G*G*I*G |
| 16 | T*C_G_A_C_G_T_C_G_T_G_I*I*I*I |
| 17 | T*C_G_A_C_G_T_C_G_T_G_G_G*G*G |
| 18 | T*C_G_A_C_G_T_C_G*T |
| 19 | A*C*G*A*C*G*T*T*T*T*G*T*C*G*T*T*T*T*G*T* |
| | C*G*T*T |
| 20 | A*C_G_A_C_G_T_C_G*T |
| 21 | A*C_G_A_C_G_T_C_G*T*T*T*T*T*T*T*T*T*T*T |
| 22 | A*C*G*A*C*G*T*C*G*T*T*T*T*T*T*T*T*T*T*T |
| 23 | G*G*G_G_T*C_G_A_C_G_T_C_G_T_G_G*G*G*G*G*G |
| 24 | G*G*G_G_T*C_G_A_C_G_T_C_G_T_G_G*G*G*G |
| 25 | G*G*G_G_T_C_G_T_C_G_T_C_G_T_G_G*G*G*G*G*G |
| 26 | G*G_G_T_G_G_G_T_G_G_G_T_G_G_G*T |
| 27 | G*G_C_G_T_G_G_C_G_T_G_G_C_G_T_G_G_C_G*T |
| 28 | G*G_C_G_T_C_G_G_C_G_T_C_G_G_C_G_T_C_G_ |
| | G_C_G*T |
| 29 | I*C_G_A_C_G_T_C_G_T_G_G*G*G*G |
| 30 | T*C_G_A_C_G_T_C_G_T_G_G_G_G_G*T |
| 31 | T_C_G_A_C_G_T_C_G_T_D_D_D_D_T_C_G_A_C_ |
| | G_T_C_G_T_D_D_D |
| 32 | T*C_G*A*C_G*T*C_G*T_G_G*G*G*G |
| 33 | T*C*G*A*C*G*T*C*G*T*G*G*G*G*G |
| 34 | A*C_G_A_C_G_T_C_G_T_G_G*G*G*G |
| 35 | T*C_G_A_C_G_A_C_G_T_G_G*G*G*G |
| 36 | A*C_G_T_C_G_T_C_G_T_G_G*G*G*G |
| 37 | T*C_G_A_C_G_T_C_G_T_C_G*G*G*G |
| 38 | T*C_G_A_C_G_T_C_G_T_G_G*T*G*G |
| 39 | T*C_G_A_C_G_T_C_G_T_G_G*G*G |
| 40 | T*C_G_A_C_G_T_C_G_T_hex |
| 41 | T*C_G_A_C_G_T_C_G_T_teg |
| 42 | T*C_G_A_C_G_T_C_G*T_Chol |
| 43 | T_C_G_A_C_G_T_C_G_T_Chol |
| 44 | Chol_T_C_G_A_C_G_T_C_G_T_Chol |
| 45 | T_C_G_A_C_G_T_C_G_T_G_G*G*G*G |
| 46 | T_C_G_A_C_G_T_C_G_T_G_G*G*G*T |
| 47 | T_C_G_A_C_G_T_C_G_A_G_G*G*G*G |
| 48 | T_C_G_A_C_G_T_C_G_A_G_G*G*G*T |
| 49 | T_C_G_A_C_G_T_C_G_A_G*G*G*G |
| 50 | T_C_G_A_C_G_T_C_G_A_chol |
| 51 | T*G*C*T*G*C*T*T*T*T*G*T*G*C*T*T*T*T*G*T* |
| | G*C*T*T |
| 52 | T*C_G*A*C_G*T*C_G*T |
| 53 | T*C_G*T*C_G*T*T*T*C_G*T*C_G*T*T_hex |
| 54 | A_C_G_A_C_G_T_C_G_T_T*T*T*T_A_C_G_A_C_ |
| | G_T_C_G_T_hex |
| 55 | T*C_G*T*C_G*T*T*T*C_G*T*C_G*T*T_teg |
| 56 | A_C_G_A_C_G_T_C_G_T_T*T*T*T_A_C_G_A_C_ |
| | G_T_C_G_T_teg |
| 57 | T*C_G*T*C_G*T*T*T*C_G*T*C_G*T*T |
| 58 | T*C_G*T*C G*T*T*T*T_G*T*C_G*T*T*T*T*G* |
| | T*C_G*T*T |
| 59 | T*C_G*T*C_G*T*T*T*T*G*T*C_G*T*T*T*T*G* |
| | T*C_G*T*T |
| 60 | T*C*G*T*C*G*T*T*T*T*G*T*C*G*T*T*T*T*G* |
| | T*C*G*T*T |
| 61 | T*C_G*T*C_G*T*T*T*T_G*T*C_G*T*T*T*T*G* |
| | T*C_G*T*T_hex |
| 62 | T*C_G*T*C_G*T*T*T*T_G*T*C_G*T*T*T*T*G* |
| | T*C_G*T*T_teg |
| 63 | T*C*G*T*C*G*T*T*T*T*C*G*G*C*G*G*C*C*G* |
| | C*C*G |
| 64 | A_C_G_A_C_G_T_C_G_T_hex |
| 65 | A_C_G_A_C_G_T_C_G_T_teg |
| 66 | A_C_G_A_C_G_T_C_G_T_D_D_D_D_A_C_G_A_C_ |
| | G_T_C_G_T_D_D_D |
| 67 | D_D_D_A_C_G_A_C_G_T_C_G_T_D_D_D_D_A_C_ |
| | G_A_C_G_T_C_G_T_D_D_D |
| 68 | T*C_G*T*C_G*A*C_G*T*T*C_G*G*C*G*C_G*C* |
| | G*C*C*G |
| 69 | T*C_G*T*C_G*A*C_G*A*T*C_G*G*C*G*C_G*C* |
| | G*C*C*G |

Key
chol cholesterol
teg triethylene glycol
hex hexadecyl glyceryl ether
phosphodiester internucleotide bond
* phosphorothioate internucleotide bond

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

1. An immunostimulatory oligonucleotide of the formula
(SEQ ID NO: 70)
5′-(Z₁)_{K}X₁Y₁R₁X₂Y₂R₂X₃Y₃R₃(Z₂)_{L}(G)_{N}(Z)_{M}-3′
wherein X₁ is any nucleotide except dG, X₂ and X₃ are any nucleotide, Y₁, Y₂ and Y₃ are dC, 5-methyl-dC, 5-hydroxy-dC or 5-fluoro-dC, R₁, R₂ and R₃ are dG, dI, 6-Thio-dG, or 7-deaza-dG, and Z₁, Z₂ and Z₃ are any nucleotide, and wherein K, L, and M each independently represent 0-10, N is 4-10 and wherein the immunostimulatory oligonucleotide is less than 16 nucleotides in length.

2. The immunostimulatory oligonucleotide of claim 1, wherein X₁ includes T, dU, dI, or dA; X₂ includes T, dU, dA or 7-deaza-dA; Z₁ includes d6, dt, dU, dI or 7-deaza-dG; Z₂ includes T and Z₃ includes T.

3. The immunostimulatory oligonucleotide of claim 1, wherein the immunostimulatory oligonucleotide includes fewer than six phosphorothioate linkages.

4. The immunostimulatory oligonucleotide of claim 1, wherein the immunostimulatory oligonucleotide comprises four phosphorothioate linkages.

5. The immunostimulatory oligonucleotide of claim 1, wherein the sequence Y₁R₁X₂Y₂R₂X₃Y₃R₃ forms a palindrome or near-palindrome.

6. The immunostimulatory oligonucleotide of claim 1, further comprising a palindromic domain of at least 6 and less than 11 nucleotides in length and including at least 3 YR dinucleotides having phosphodiester or phosphodiester-like internucleotide linkages, wherein Y is dC, 5-methyl-dC, 5-hydroxy-dC or 5-fluoro-dC, and R is dG, dI, 6-Thio-dG, or 7-deaza-dG, linked to a Poly G domain, either directly or indirectly, wherein the Poly G domain includes at least 3 and less than 8 consecutive Gs, wherein when the palindromic domain is indirectly linked to the Poly-G domain, the indirect linkage is comprised of a nucleotide sequence of 1-10 nucleotides or a non-nucleotide linker, wherein the oligonucleotide has a length of less than 18 nucleotides.

7. The immunostimulatory oligonucleotide of claim 6, wherein the oligonucleotide includes at least 2 and less than 6 stabilized internucleotide linkages.

8. The immunostimulatory oligonucleotide of claim 6, wherein the stabilized internucleotide linkages are phosphorothioate linkages.

9. The immunostimulatory oligonucleotide of claim 6, wherein each nucleotide of the palindromic domain has a phosphodiester internucleotide linkage.

10. The immunostimulatory oligonucleotide of claim 1, further comprising a palindromic domain of at least 6 and less than 11 nucleotides in length and including at least 3 Y′R′ dinucleotides having phosphodiester or phosphodiester-like internucleotide linkages, wherein Y′ is 5-methyl-dC, 5-hydroxy-dC or 5-fluoro-dC, and R is dI, dG, 6-Thio-dG, or 7-deaza-dG, linked to a Poly G domain, either directly or indirectly, wherein the Poly G domain includes at least 3 and less than 8 consecutive Gs, wherein when the palindromic domain is indirectly linked to the Poly-G domain, the indirect linkage is comprised of a nucleotide sequence of 1-10 nucleotides or a non-nucleotide linker.

11. An immunostimulatory oligonucleotide of the formula
(SEQ ID NO: 71)
5′-(Z₁)_{K}X₁Y₁R₁X₂Y₂R₂X₃Y₃R₃(Z₂)_{L}Q-3′
wherein X₁ is any nucleotide except dG, X₂ and X₃ are any nucleotide, Y₁ and Y₂ are dC, 5-methyl-dC, 5-hydroxy-dC or 5-fluoro-dC, R₁, R₂ and R₃ are dG, dI, 6-Thio-dG, or 7-deaza-dG, and Z₁ and Z₂ are any nucleotide, and Q is a lipophilic moiety, and wherein K, L, and M each independently represent 0-10, N is 4-10 and wherein the immunostimulatory oligonucleotide is less than 16 nucleotides in length.

12. A composition comprising the immunostimulatory oligonucleotide of any one of claim 1 and a pharmaceutical carrier.

13. The composition of claim 1 wherein the immunostimulatory oligonucleotide sequence includes SEQ ID NO:3; SEQ ID NO:4; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO.15; SEQ ID NO:16; SEQ ID NO:17; SEQ ID NO:18; SEQ ID NO:29, SEQ ID NO:30 SEQ ID NO:34; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:45; SEQ ID NO:46; SEQ ID NO:47; SEQ ID NO:48 SEQ ID NO:49.

14. A method of stimulating an immune response in a subject, comprising administering to a subject in need of such treatment the composition of claim 1.

15. The method of claim 1, wherein the subject in need has or is at risk of having cancer, infectious disease, asthma, allergy, allergic rhinitis, or autoimmune disease.
